(19) United States
(12) Patent Application Publication
(10) Pub. No.: US 2026/0354971 A1
(43) Pub. Date: Nov. 12, 2026

(54) DELIVERY APPARATUS FOR A PROSTHETIC HEART VALVE

(71) Applicant: Edwards Lifesciences Corporation, Irvine, CA (US)

(72) Inventors: Grant Jason Gangeness, Carlsbad, CA (US); Tri D. Tran, Fountain Valley, CA (US); Kim Le, Carlsbad, CA (US); Andrew Steven Roundy, Laguna Hills, CA (US); Domonique Bliss Suntay, Irvine, CA (US)

(21) Appl. No.: 19/706,694

(22) Filed: Jun. 12, 2026

Related U.S. Application Data
(63) Continuation of application No. PCT/US2024/058924, filed on Dec. 6, 2024.
(60) Provisional application No. 63/613,172, filed on Dec. 21, 2023.

(51) Int. Cl. A61F 2/24

(52) U.S. Cl. CPC A61F 2/2436

(57) ABSTRACT

A delivery apparatus can comprise a delivery shaft with a first shaft and a second shaft. A distal region of the first shaft can comprise a distal tip portion configured to engage an implant apparatus supported by the second shaft and having a distal tip portion hardness; a first flex portion extending on a proximal side of the distal tip portion and having a first flex portion hardness that is less than the distal tip portion hardness; a second flex portion extending on a proximal side of the first flex portion and having a second flex portion hardness that is greater than the first flex portion hardness; and a distal region bulk portion extending between the second flex portion and the proximal region and having a distal region bulk hardness that is greater than the second flex portion hardness.

### CROSS-REFERENCE TO RELATED APPLICATION

This application is a continuation of PCT Patent Application No. PCT/US2024/058924, filed on Dec. 6, 2024 which claims priority to and the benefit of U.S. Provisional Patent Application No. 63/613,172, filed on Dec. 21, 2023, each of these applications being incorporated by reference herein in its entirety.

### FIELD

The present disclosure relates to delivery apparatus for prosthetic implants, including delivery apparatus and/or catheters for delivering prosthetic implants to a target implantation site.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing an implant delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The implant delivery apparatus can be inserted through a lumen of the guide catheter to help direct the implant delivery apparatus to a target implantation site (e.g., a native valve region) within the patient and/or help position the implant delivery apparatus at the target implantation site.

### SUMMARY

Described herein are prosthetic heart valves, docking devices, delivery apparatuses, guide catheters, systems including the guide catheters, and methods for implanting docking devices and prosthetic heart valves. The disclosed delivery apparatus can, for example, be configured to transport a prosthetic medical device through a main lumen of a guide catheters in order to introduce the prosthetic medical device into a patient's vasculature and guide the prosthetic medical device toward a target implantation site. In some examples, a delivery apparatus can include a shaft with a distal region that includes flex portions of varying hardness to facilitate flexure of the shaft when inserted into a guide catheter. As such, the devices and methods disclosed herein can, among other things, overcome one or more of the deficiencies of typical delivery apparatus.

A delivery apparatus for a prosthetic implant can comprise a handle and one or more shafts coupled to the handle.

In some examples, a delivery apparatus comprises a first shaft comprising a distal region and a proximal region and a second shaft disposed radially interiorly of the first shaft.

In some examples, the distal region of the first shaft comprises a distal tip portion configured to engage an implant apparatus supported by the second shaft and having a distal tip portion hardness, a first flex portion extending on a proximal side of the distal tip portion and having a first flex portion hardness that is less than the distal tip portion hardness, a second flex portion extending on a proximal side of the first flex portion and having a second flex portion hardness that is greater than the first flex portion hardness, and a distal region bulk portion extending between the second flex portion and the proximal region and having a distal region bulk hardness that is greater than the second flex portion hardness.

In some examples, the distal region of the first shaft further comprises a third flex portion extending on a proximal side of the second flex portion and having a third flex portion hardness that is greater than the second flex portion hardness.

In some examples, the distal region of the first shaft further comprises a fourth flex portion extending on a proximal side of the third flex portion and having a fourth flex portion hardness that is greater than the third flex portion hardness.

In a representative example, a delivery apparatus comprises a delivery shaft comprising a first shaft comprising a distal region and a proximal region and a second shaft disposed radially interiorly of the first shaft. The first shaft is configured to translate relative to the second shaft along a longitudinal axis of the delivery shaft. The second shaft comprises a distal end region configured to support a prosthetic implant thereon. The distal region of the first shaft comprises a distal tip portion configured to engage an implant apparatus supported by the second shaft and having a distal tip portion hardness, a first flex portion extending on a proximal side of the distal tip portion and having a first flex portion hardness that is less than the distal tip portion hardness, a second flex portion extending on a proximal side of the first flex portion and having a second flex portion hardness that is greater than the first flex portion hardness, and a distal region bulk portion extending between the second flex portion and the proximal region and having a distal region bulk hardness that is greater than the second flex portion hardness.

In a representative example, a delivery apparatus comprises a handle and a delivery shaft extending distally from the handle. The delivery shaft comprises a first shaft comprising a distal region and a proximal region and a second shaft disposed radially interiorly of the first shaft. The first shaft is configured to translate relative to the second shaft along a longitudinal axis of the delivery shaft. The second shaft comprises a distal end region configured to support a prosthetic implant thereon. The handle is free of articulation members for bending a distal end portion of the delivery shaft.

In some examples, a delivery apparatus comprises one or more of the components recited in Examples 1-68, Examples 72-73, or Examples 81-84 below.

A shaft for a delivery apparatus can comprise a lumen configured to receive a second shaft therein and a distal region comprising a distal tip portion, a first flex portion, a second flex portion, and a distal region bulk portion.

In some examples, the distal tip portion is configured to engage an implant apparatus supported by the second shaft and has a distal tip portion hardness.

In some examples, the first flex portion extends on a proximal side of the distal tip portion and has a first flex portion hardness that is less than the distal tip portion hardness.

In some examples, the second flex portion extends on a proximal side of the first flex portion and has a second flex portion hardness that is greater than the first flex portion hardness.

In some examples, the distal region bulk portion extends between the second flex portion and the proximal region and has a distal region bulk hardness that is greater than the second flex portion hardness.

In a representative example, a shaft comprises a distal region, a proximal region, and a lumen configured to receive a second shaft therein. The distal region of the shaft comprises a distal tip portion configured to engage an implant apparatus supported by the second shaft and having a distal tip portion hardness, a first flex portion extending on a proximal side of the distal tip portion and having a first flex portion hardness that is less than the distal tip portion hardness, a second flex portion extending on a proximal side of the first flex portion and having a second flex portion hardness that is greater than the first flex portion hardness, and a distal region bulk portion extending between the second flex portion and the proximal region and having a distal region bulk hardness that is greater than the second flex portion hardness.

In some examples, a shaft comprises one or more of the components recited in Examples 65-67 below.

A delivery assembly can comprise a guide catheter and a delivery apparatus.

In some examples, a guide catheter comprises a guide catheter handle and a guide catheter shaft extending distally from the guide catheter handle.

In some examples, a delivery apparatus comprises a delivery apparatus handle and a delivery shaft extending distally from the delivery apparatus handle.

In some examples, the guide catheter handle comprises one or more articulation members configured to be adjusted by a user to articulate a distal end portion of the guide catheter shaft within the patient's body, and the delivery apparatus handle lacks articulation members for articulating a distal end portion of the delivery shaft.

In some examples, a delivery assembly comprises a docking device delivery apparatus configured to deliver a docking device to the implantation site.

In some examples, a docking device delivery apparatus comprises a docking device delivery handle, a docking device delivery shaft extending distally from the handle and configured to be advanced through the guide catheter shaft, and a pusher assembly configured to deploy the docking device at the implantation site.

In some examples, a delivery assembly comprises a prosthetic implant.

In a representative example, a delivery assembly comprises a guide catheter comprising a guide catheter handle and a guide catheter shaft extending distally from the guide catheter handle and a delivery apparatus comprising a delivery apparatus handle and a delivery shaft extending distally from the delivery apparatus handle. The delivery shaft is configured to be advanced through the guide catheter shaft to deliver a prosthetic implant to an implantation site within a patient's body. The guide catheter handle comprises one or more articulation members configured to be adjusted by a user to articulate a distal end portion of the guide catheter shaft within the patient's body. The delivery apparatus handle lacks articulation members for articulating a distal end portion of the delivery shaft.

In some examples, a delivery assembly comprises one or more of the components recited in Examples 74-80 or Examples 85-90 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a perspective view of an exemplary delivery apparatus for a prosthetic heart valve.

FIG. 6 is a perspective view of the implantable prosthetic valve and the delivery apparatus of FIG. 5 with an outer shaft in an extended configuration.

FIG. 7 is a perspective view of the implantable prosthetic valve and the delivery apparatus of FIG. 6 with the outer shaft in a retracted configuration.

FIG. 8 is a cross-sectional view of a delivery apparatus extending within a guide catheter according to an example.

FIG. 9 is a cross-sectional view of a first shaft of a delivery apparatus according to an example.

FIG. 10 is a cross-sectional view of the first shaft of FIG. 9 as viewed along the line 10-10 in FIG. 9.

FIG. 11 is a table representing construction details of three exemplary designs of a first shaft of a delivery apparatus.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a delivery apparatus (sometimes referred to as a catheter) that can be used to navigate a subject's vasculature to deliver an implantable, expandable medical device (e.g., a prosthetic heart valve), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

As introduced above, a guide catheter can be inserted into a patient's vasculature and then receive an implant delivery apparatus within a main lumen of the guide catheter in order to direct the delivery apparatus therethrough to a target implantation site for a prosthetic implant. In some examples, an inner diameter of the main lumen of the guide catheter and an outer diameter of portions of the implant delivery apparatus can be closely and/or approximately matched. In some examples, however, the outer diameter of portions of the implant delivery apparatus can be slightly less than the inner diameter of the main lumen of the guide catheter, which can cause the implant delivery apparatus to buckle slightly within the main lumen as the implant delivery apparatus is advanced through the guide catheter. In some examples, this can yield a buildup of friction within the guide catheter that is abruptly released as the prosthetic implant exits the guide catheter, causing the prosthetic implant to “jump” out of the guide catheter. Accordingly, improvements to the delivery apparatus that decrease or prevent the incidence and/or severity of such valve jumping are desirable. Such improvements can, for example, assist in advancing the delivery apparatus through the guide catheter with a constant push force and in ejecting the prosthetic implant from the guide catheter in a controlled manner.

Described herein are various systems, apparatuses, methods, or the like, that, in some examples, can be used in or with delivery apparatuses for prosthetic medical devices (such as prosthetic heart valves or docking devices). In some examples, such systems, apparatuses, and/or methods can provide a close correspondence between an inner diameter of the main lumen of the guide catheter and an outer diameter of portions of the implant delivery apparatus to reduce an incidence of buckling of the implant delivery apparatus within the guide catheter. As a result, the implant delivery apparatus can be advanced through the guide catheter at a readily controllable rate and/or with a constant push force applied to the implant delivery apparatus.

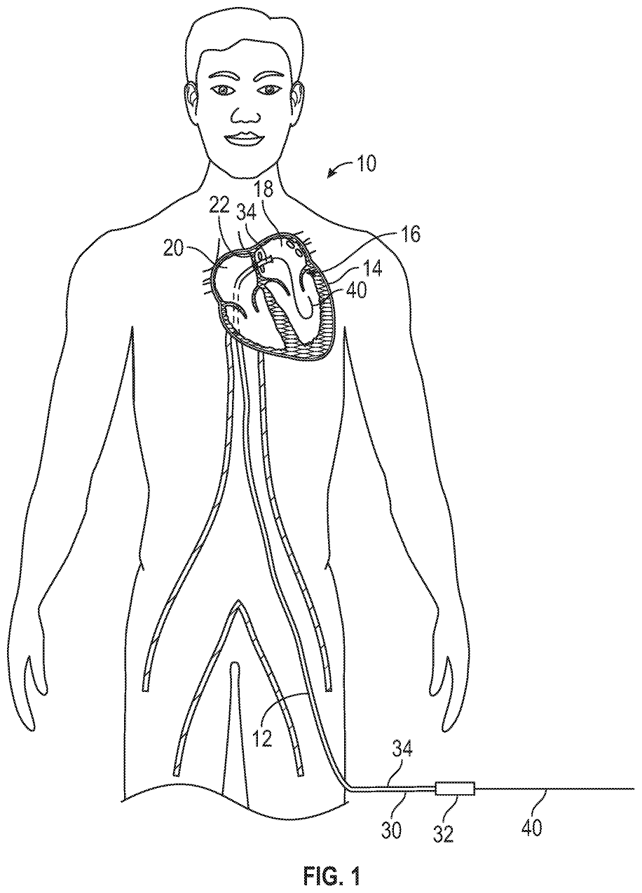

In some examples, the delivery apparatuses (or implant catheters) and guide catheters disclosed herein can be introduced into the vasculature of a patient and guide the one or more delivery apparatuses at least partially through the vasculature toward a target implantation site. For example, FIGS. 1-4 schematically illustrate an exemplary transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus. The prosthetic heart valve delivery apparatus is used to deliver a transcatheter prosthetic heart valve inside the docking device.

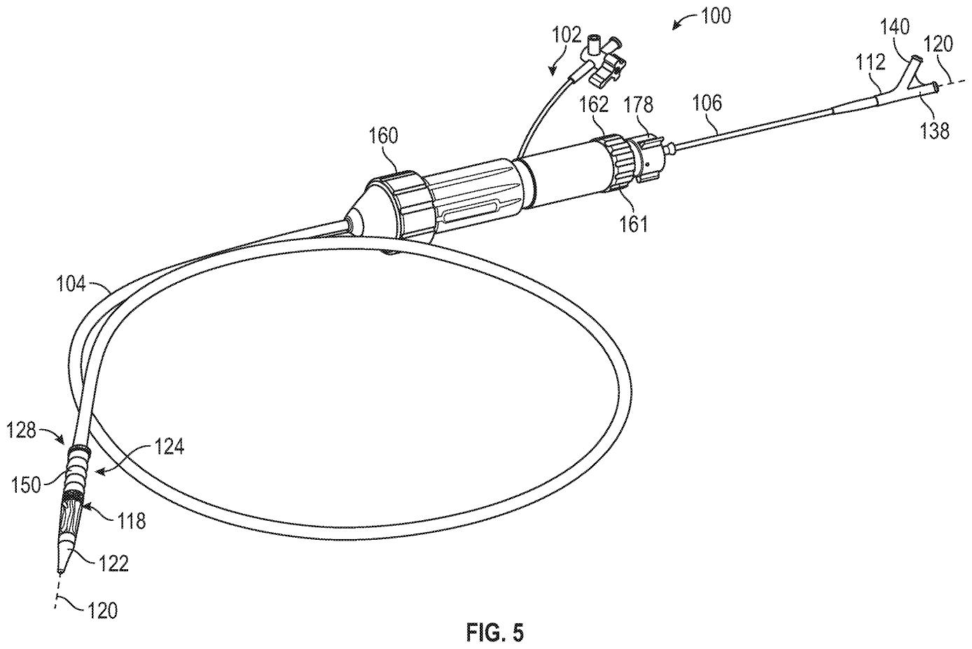

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve. An exemplary delivery apparatus for delivery a prosthetic heart valve within a docking device at a native heart valve is shown in FIGS. 5-10.

### Examples of the Disclosed Technology

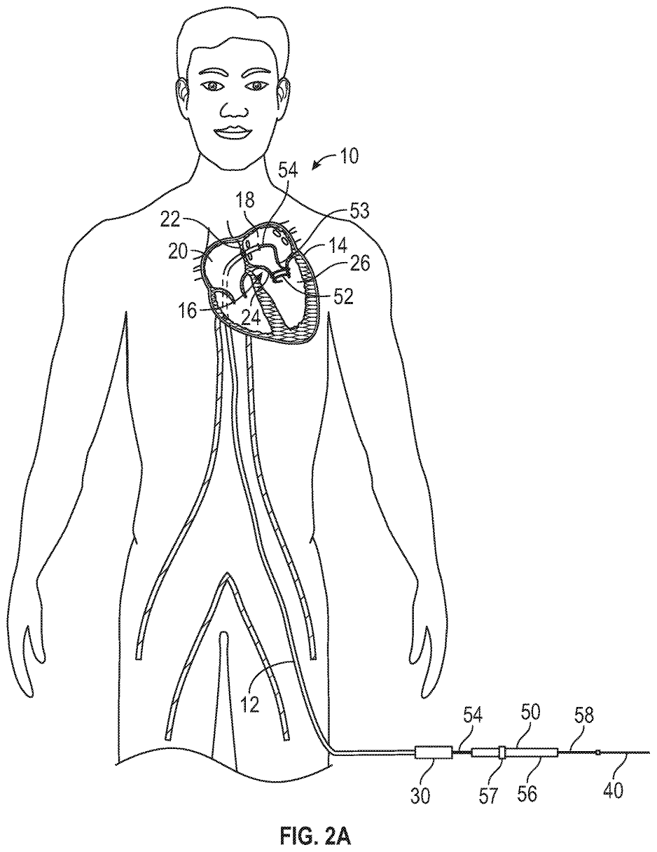

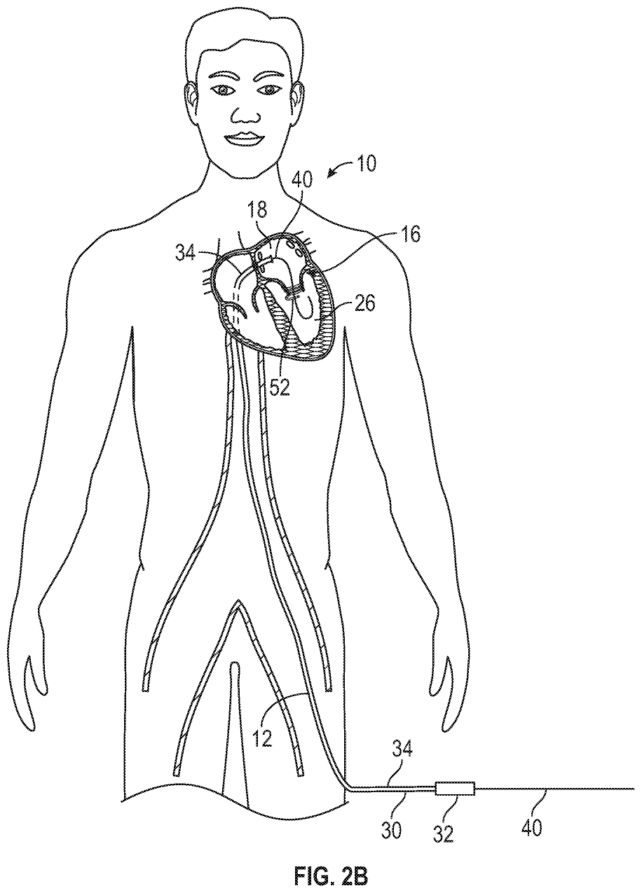

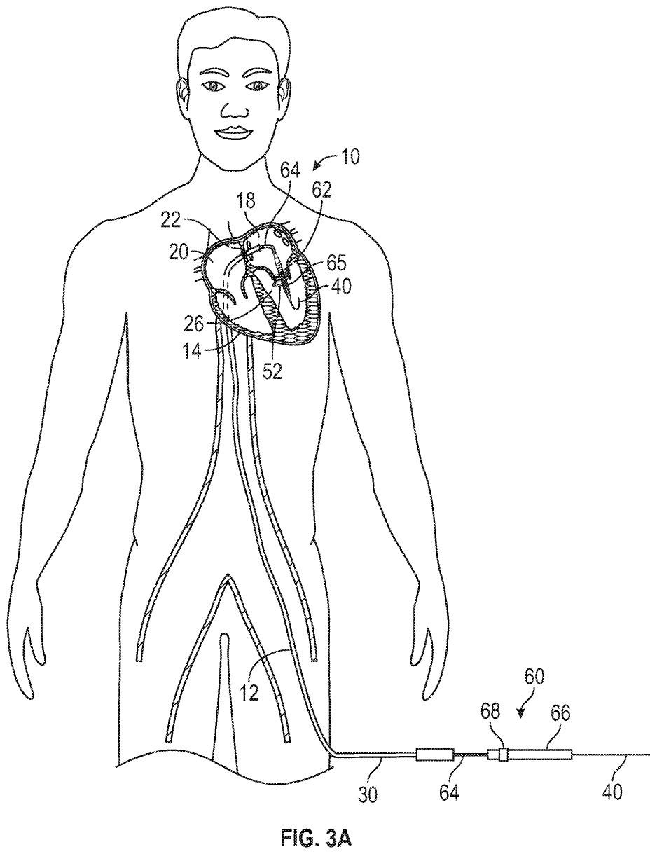

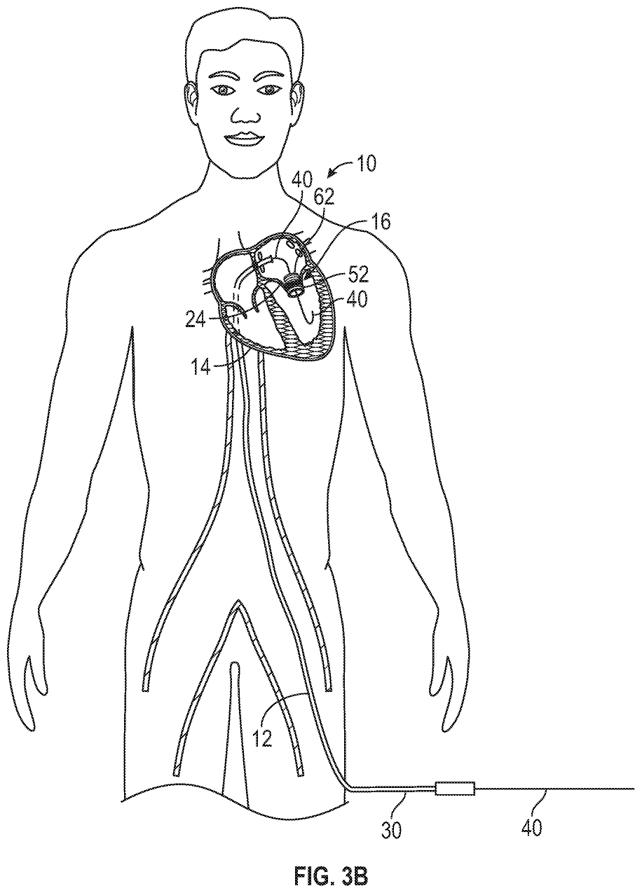

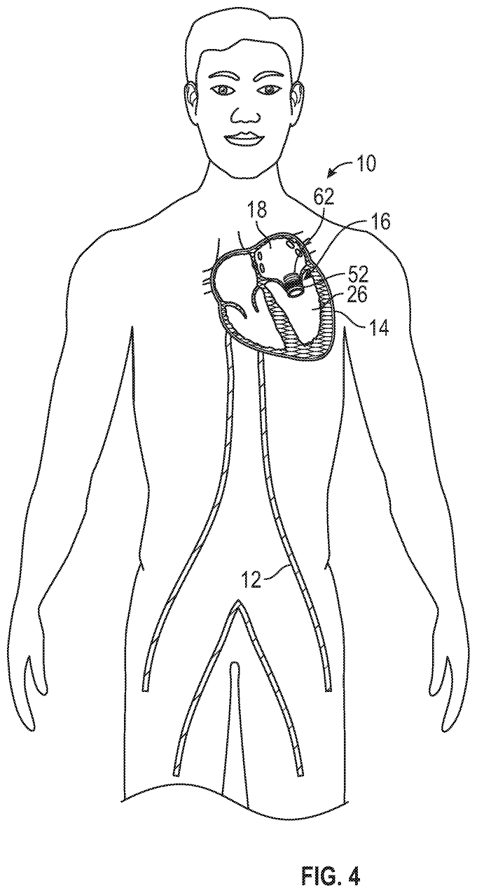

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device”, “introducer”, or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can make a small incision in an atrial septum 22 of the heart 14 to allow access to the left atrium 18 from the right atrium 20. The user can insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In other examples, the handle 66 can omit and/or be free of such articulation members for articulating the distal end of the delivery shaft 64. In particular, in some examples, the delivery shaft 64 can be navigated and/or guided through the patient's vasculature by the guide catheter 30, such as by manipulating the handle 32 to flex, bend, twist, turn, and/or otherwise articulate a distal end portion of the guide catheter 30. With the guide catheter 30 shaped and/or positioned to terminate at or near the implantation site, advancing the delivery shaft 64 through the guide catheter 30 can serve to navigate the prosthetic heart valve 62 toward the implantation site without requiring independent articulation of the delivery shaft 64 by the user.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In some examples, the prosthetic heart valve 62 can be self-expanding and can be configured to radially expand on its own upon removable of a sheath or capsule covering the radially compressed prosthetic heart valve 62 on the distal end portion of the delivery shaft 64.

In some examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14. Alternatively, in an example in which the handle 66 lacks such articulation members, the user can advance the delivery shaft 64 through the guide catheter 30 such that the delivery shaft 64 follows the path established by the guide catheter 30.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and remove the delivery shaft 54 of the docking device delivery apparatus 50 from the patient 10. The user may advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may expand the prosthetic heart valve 62 within the docking device 52 before removing the prosthetic valve delivery apparatus 60 from the patient 10. In some examples, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and through the aortic valve into the left ventricle 26.

FIG. 5 illustrates an exemplary prosthetic heart valve delivery apparatus 100 (which can also be referred to here as an “implant catheter”) that can be used in lieu of the prosthetic valve delivery apparatus 60 of FIG. 3A to implant an expandable prosthetic heart valve. In some examples, the delivery apparatus 100 is specifically adapted for use in introducing a prosthetic heart valve into a heart.

The delivery apparatus 100 in the illustrated example of FIG. 5 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. In some examples, the delivery apparatus 100 can further comprise an inner shaft extending distally from the handle 102 coaxially through the intermediate shaft 106 and the outer shaft 104 and proximally from the handle 102 coaxially through the intermediate shaft.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion that extends proximally from a proximal end of the handle 102, to an adaptor 112. The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

In some examples, the intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft can extend distally beyond the distal end portion of the intermediate shaft 106 toward or to a nose cone 122 at a distal end of the delivery apparatus 100.

In some examples, a distal end of the balloon 118 can be coupled to a distal end of the delivery apparatus 100, such as to the nose cone 122 (as shown in FIG. 5), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 (shown in FIG. 5) can overly a distal shoulder of the delivery apparatus 100. As shown in FIG. 5, a prosthetic heart valve 150 can be mounted around the balloon 118, at the valve mounting portion 124 of the delivery apparatus 100, in a radially compressed state. The prosthetic heart valve 150 can be configured to be radially expanded by inflation of the balloon 118 at a native valve annulus, as described above with reference to FIGS. 3A and 3B.

A balloon shoulder assembly of the delivery apparatus 100, which includes the distal shoulder, is configured to maintain the prosthetic heart valve 150 (or other medical device) at a fixed position on the balloon 118 during delivery through the patient's vasculature.

The outer shaft 104 can include a distal tip portion 128 mounted on its distal end. In some examples, the outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 5) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

In other examples, the handle 102 can omit and/or be free of one or more such steering members for articulating the distal end of the delivery apparatus 100. In particular, in some examples, the delivery apparatus 100 can be navigated and/or guided through the patient's vasculature by a guide catheter (e.g., the guide catheter 30 of FIGS. 1-3B), such as by manipulating a handle of the guide catheter (e.g., the handle 32 of FIGS. 1-2B) to flex, bend, twist, turn, and/or otherwise articulate a distal end portion of the guide catheter. With the guide catheter shaped and/or positioned to terminate at or near the implantation site, advancing the delivery apparatus 100 through the guide catheter 30 can serve to navigate the prosthetic valve 150 toward the implantation site without requiring independent articulation of the delivery apparatus 100 by the user.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site).

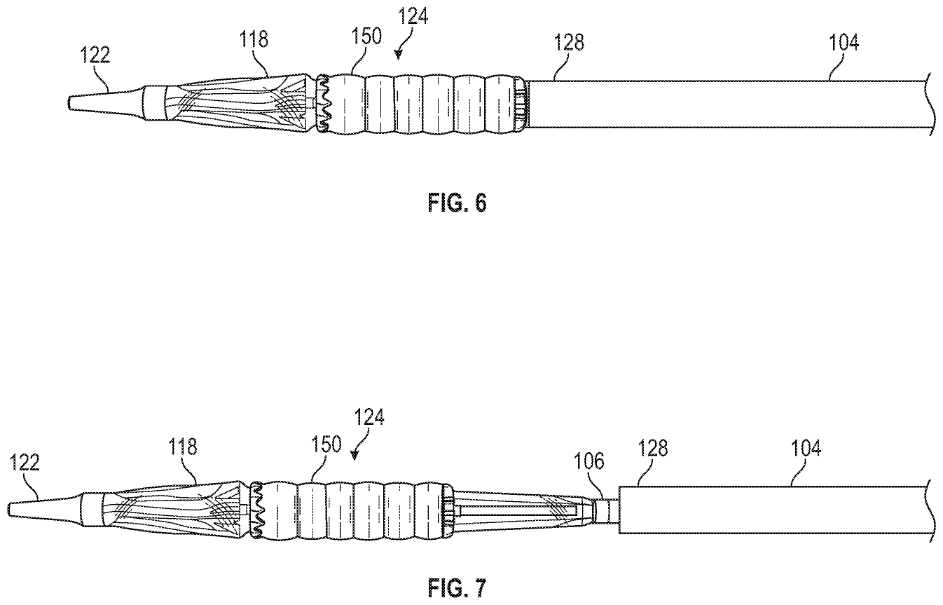

As discussed above, the outer shaft 104 and the intermediate shaft 106 can be configured to translate longitudinally relative to one another. As an example, FIGS. 6-7 illustrate a manner in which the outer shaft 104 can be moved (e.g., retracted) relative to the intermediate shaft 106 during a mitral valve replacement procedure. This may be done, for example, to expose a full length of the balloon 118 prior to inflating the balloon 118.

To expose the balloon 118 and/or the prosthetic valve 150 near the implantation site, a mitral valve replacement procedure can include advancing the delivery apparatus 100 through the guide catheter and/or withdrawing the guide catheter relative to the delivery apparatus 100 to remove the prosthetic valve 150 from the guide catheter. With reference to the mitral valve replacement procedure described above in combination with FIGS. 1-4, such a step can correspond to advancing from the configuration of FIG. 2B to the configuration of FIG. 3A (with FIG. 3A additionally showing the inflatable balloon fully free of the delivery apparatus in the manner of FIG. 7).

In some instances, urging the delivery apparatus 100 in a distal direction through the guide catheter can produce an accumulation of static frictional forces between the prosthetic valve 150 and an inner surface of the guide catheter. Additionally or alternatively, the outer shaft 104 can have an outer diameter that is slightly less than an inner diameter of the guide catheter through which the outer shaft 104 extends. In such examples, urging the delivery apparatus 100 in a distal direction through the guide catheter can result in a bending or “snaking” of the outer shaft 104 within the guide catheter, producing an accumulation of potential energy. As a result, when the prosthetic valve 150 is urged free of the guide catheter, the prosthetic valve 150 can exit the guide catheter abruptly and/or at a discontinuous rate as the frictional forces and/or potential energy are rapidly released, potentially resulting a “jump” of the prosthetic valve 150 relative to the guide catheter.

By contrast, the delivery apparatus of the present disclosure can be configured to yield a more controlled exit of the prosthetic valve 150 from the guide catheter during the implantation procedure. In particular, and as described in more detail below, delivery apparatuses according to the present disclosure can include an outer shaft with an outer diameter that closely corresponds to an inner diameter of a guide catheter, thus promoting improved coaxiality between the outer shaft and the guide catheter during the implantation procedure. Additionally, delivery apparatuses according to the present disclosure can include an outer shaft with a distal region formed of materials exhibiting progressively varying degrees of hardness, which can facilitate flexure of the distal region of the outer shaft. For example, the distal region of the outer shaft can be configured to exhibit varying degrees of material hardness to facilitate tracking of the outer shaft through a flexed section of the guide catheter. With reference to the mitral valve replacement procedure described above, this can promote coaxiality between the delivery shaft 64 and the docking device 52 upon exit of the prosthetic heart valve 62 from the shaft 34 of the guide catheter 30.

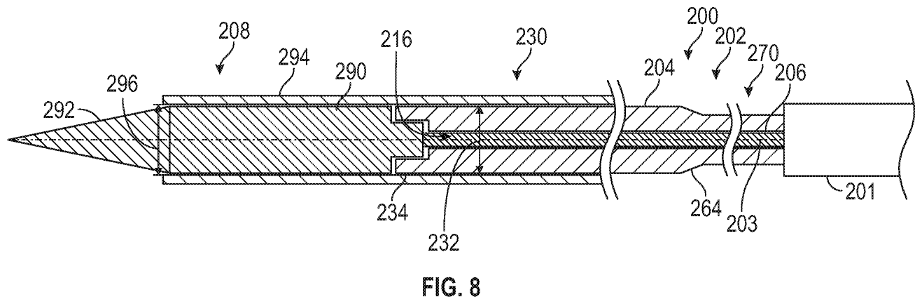

FIG. 8 illustrates an exemplary prosthetic heart valve delivery apparatus (which can also be referred to here as an “implant catheter”) that can be used in lieu of the prosthetic valve delivery apparatus 60 of FIG. 3A and/or the prosthetic heart valve delivery apparatus 100 of FIGS. 5-7 to implant an expandable prosthetic heart valve. In some examples, the delivery apparatus of FIG. 8 is specifically adapted for use in introducing a prosthetic heart valve into a heart.

Specifically, FIG. 8 is a cross-sectional view of a delivery apparatus 200 including a delivery shaft 202 extending within a shaft 294 of a guide catheter. The delivery shaft 202 includes a first shaft 204 with a distal region 230 and a proximal region 270 and a second shaft 206 disposed radially interiorly of the first shaft 204. The first shaft 204 also may be referred to as an outer shaft 204. The second shaft 206 also may be referred to as an intermediate shaft 206, an inner shaft 206, and/or a balloon shaft 206. The first shaft 204 is configured to translate relative to the second shaft 206 along a longitudinal axis 203 of the delivery shaft 202. Similarly, the delivery shaft 202 (e.g., the first shaft 204 and/or the second shaft 206) is configured to translate relative to the shaft 294 along the longitudinal axis 203. A proximal portion of the delivery shaft 202 (e.g., the proximal region 270 of the first shaft 204 and/or a corresponding region of the second shaft 206) can extend to and/or through a handle 201 of the delivery apparatus 200, such as the handle 102 of FIG. 5.

The handle 201 can include and/or be any of a variety of structures and/or mechanisms for manipulating the delivery shaft 202, such as for translating the first shaft 204 and/or the second shaft 206 relative to one another and/or relative to the shaft 294 of the guide catheter. For example, the handle 201 can include any components and/or features discussed herein with reference to the handle 102 of FIG. 5. In particular, in some examples, the handle 201 can include one or more physically adjustable control members that are configured to be adjusted by the user to flex, bend, twist, turn, and/or otherwise articulate a distal end portion of the delivery shaft 202 to aid in navigating the delivery shaft 202 through the patient's vasculature. In other examples, the handle 201 can omit and/or be free of such articulation members for articulating the distal end of the delivery shaft 202. In particular, in some examples, the delivery shaft 202 can be navigated and/or guided through the patient's vasculature by the shaft 294 of the guide catheter without requiring independent articulation of the delivery shaft 202 by the user.

The second shaft 206 includes a distal end region 208 configured to support an implant apparatus 290 thereon. For example, the implant apparatus 290 can include and/or be a prosthetic implant, such as the prosthetic heart valve 62 and/or the prosthetic valve 150. Additionally or alternatively, the implant apparatus 290 can include and/or be an inflatable balloon on which the prosthetic implant is crimped and/or mounted, such as the inflatable balloon 118.

In some examples, the delivery shaft 202 additionally can include an inner shaft extending distally from the handle 201 of the delivery apparatus 200 coaxially through the first shaft 204 and the second shaft 206 and proximally from the handle coaxially through the second shaft 206. A distal region of of the inner shaft can extend distally beyond the distal end region 208 of the second shaft 206 toward or to a nose cone 292 at a distal end of the delivery apparatus.

The distal region 230 of the first shaft 204 can include a distal tip portion 234 configured to engage the implant apparatus 290. In some examples, the distal tip portion 234 can be configured to resist movement of a portion of the implant apparatus 290 (e.g., the prosthetic valve) relative to another portion of the implant apparatus (e.g., the inflatable balloon) and/or relative to the second shaft 206 when the distal tip portion 234 is arranged adjacent to a proximal side of the implant apparatus 290.

As shown in FIG. 8, the radial dimensions of the first shaft 204 can closely correspond to the radial dimensions of the shaft 294 of the guide catheter. Specifically, at least a portion of the first shaft 204 (e.g., at least a distal region thereof) can have an outer diameter 232 that is approximately equal to an inner diameter 296 of the shaft 294. Such a configuration may facilitate maintaining the first shaft 204 and the shaft 294 in a coaxial orientation relative to one another as the delivery shaft 202 is advanced through the shaft 294, such as by reducing a space between the first shaft 204 and the shaft 294 within which the first shaft 204 otherwise could exhibit bending or snaking. Additionally, the first shaft 204 may be constructed with a wall thickness that is sufficiently large to restrict undesirable bending and/or kinking of the first shaft 204 within the shaft 294.

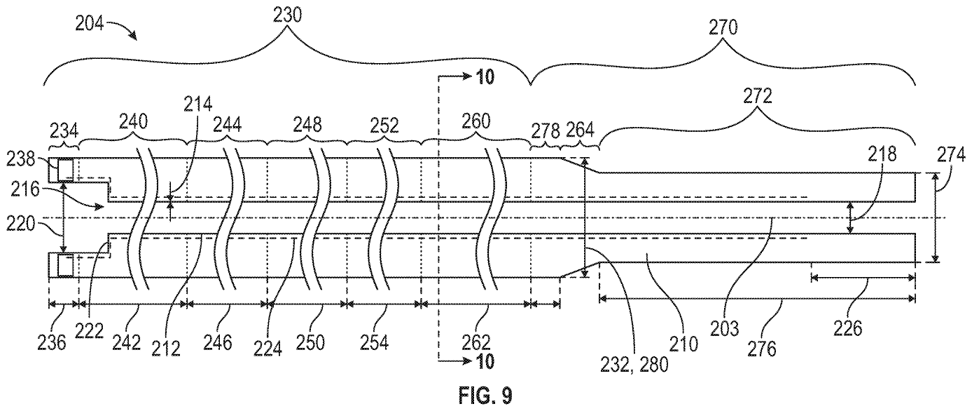

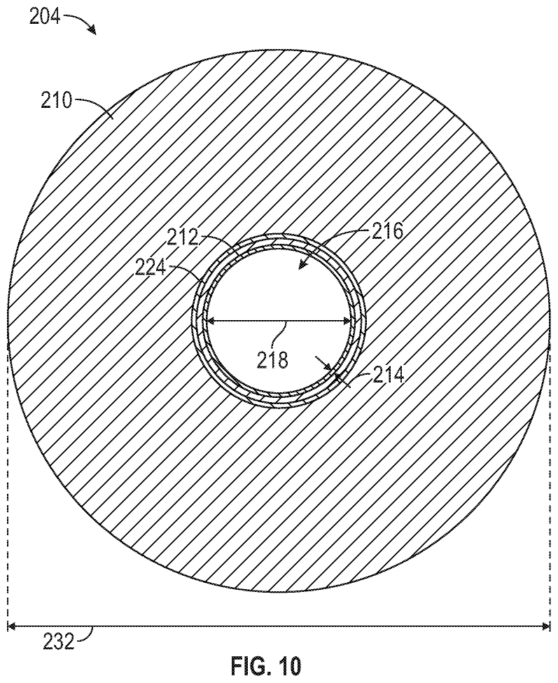

FIG. 9 is a cross-sectional view of the first shaft 204 illustrating examples of various regions of the first shaft 204 and corresponding properties thereof. The cross-sectional view of FIG. 9 represents a cross-sectional cut plane that includes a longitudinal axis 203 of the delivery shaft 202. For clarity, FIG. 9 omits cross-sectional hatch lines. As shown in FIG. 9, the distal region 230 of the first shaft 204 includes a first flex portion 240 extending on a proximal side of the distal tip portion 234 and a second flex portion 244 extending on a proximal side of the first flex portion 240. The first flex portion 240 can have a first flex portion hardness that is less than a distal tip portion hardness of the distal tip portion 234, while the second flex portion 244 can have a second flex portion hardness that is greater than the first flex portion hardness. The distal region 230 further can include a distal region bulk portion 260 extending between the second flex portion 244 and the proximal region 270. The distal region bulk portion 260 can have a distal region bulk hardness that is greater than the second flex portion hardness.

In some examples, and as shown in FIG. 9, the distal region 230 of the first shaft 204 additionally includes a third flex portion 248 extending on a proximal side of the second flex portion 244 and/or a fourth flex portion 252 extending on a proximal side of the third flex portion 248. The third flex portion 248 can have a third flex portion hardness that is greater than the second flex portion hardness, while the fourth flex portion 252 can have a fourth flex portion hardness that is greater than the third flex portion hardness. Each of the third flex portion hardness and/or the fourth flex portion hardness can be less than the distal region bulk hardness.

The first flex portion hardness, the second flex portion hardness, the third flex portion hardness, and the fourth flex portion hardness each may assume any of a variety of values. As examples, the first flex portion hardness can correspond to a durometer value that is at least 10 D, at least 20 D, at least 30 D, at least 40 D, at most 50 D, at most 35 D, at most 25 D, at most 15 D, 10-25 D, 20-35 D, 30-50 D, 10-35 D, 20-50 D, and/or 10-50 D.

Additionally or alternatively, the second flex portion hardness can correspond to a durometer value that is at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and/or 20-80 D.

Additionally or alternatively, the third flex portion hardness can correspond to a durometer value that is at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and/or 20-80 D.

Additionally or alternatively, the fourth flex portion hardness can correspond to a durometer value that is at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and/or 20-80 D.

In some examples, such as in examples in which the first shaft 204 lacks the third flex portion 248 and the fourth flex portion 252, the distal tip portion hardness can correspond to a durometer value that is 40-70 D, the first flex portion hardness can correspond to a durometer value that is 20-50 D, the second flex portion hardness can correspond to a durometer value that is 40-80 D, and the distal region bulk hardness can correspond to a durometer value that is 60-80 D. As more specific examples, the distal tip portion hardness can correspond to a durometer value that is 50-60 D, the first flex portion hardness can correspond to a durometer value that is 20-35 D, the second flex portion hardness can correspond to a durometer value that is 50-65 D, and the distal region bulk hardness can correspond to a durometer value that is 65-80 D.

In some examples, such as in examples in which the first shaft 204 includes the third flex portion 248 and the fourth flex portion 252, the distal tip portion hardness can correspond to a durometer value that is 40-70 D, the first flex portion hardness can correspond to a durometer value that is 20-40 D, the second flex portion hardness can correspond to a durometer value that is 20-45 D, the third flex portion hardness can correspond to a durometer value that is 30-65 D, the fourth flex portion hardness can correspond to a durometer value that is 40-80 D, and the distal region bulk hardness can correspond to a durometer value that is 60-80 D. As more specific examples, the distal tip portion hardness can correspond to a durometer value that is 50-60 D, the first flex portion hardness can correspond to a durometer value that is 20-30 D, the second flex portion hardness can correspond to a durometer value that is 30-40 D, the third flex portion hardness can correspond to a durometer value that is 45-60 D, the fourth flex portion hardness can correspond to a durometer value that is 50-70 D, and the distal region bulk hardness can correspond to a durometer value that is 65-80 D.

In this manner, the first flex portion 240, the second flex portion 244, the third flex portion 248, and the fourth flex portion 252 can represent portions of the distal region 230 of the first shaft 204 with respective varying material hardness that decrease when proceeding in a distal direction along such regions. Such a configuration can facilitate flexure of the distal region 230 of the first shaft 204, such as to facilitate navigating the distal region 230 through the patient's vasculature toward the implantation site. Such a configuration also may facilitate navigating the delivery shaft 202 through curves and/or bends of the shaft 294 of the guide catheter without requiring that the shape of the distal region 230 be independently controlled. For example, configuring the first shaft 204 to have a relatively low hardness toward the distal end thereof can allow for the delivery shaft 202 to follow the contours of the shaft 294 without manipulating articulation members of the handle 201 of the delivery apparatus 200 to bend the delivery shaft 202. In this manner, the delivery apparatus 200 can operate as a non-steerable (e.g., not independently steerable) delivery apparatus when the delivery shaft 202 is advanced through the shaft 294 of a steerable guide catheter.

While the reduced material hardness of the distal region of the first shaft 204 may be desirable for augmenting the flexibility thereof, it also may be desirable that the distal tip portion hardness of the distal tip portion 234 be sufficiently high to resist motion of the implant apparatus 290 in the proximal direction when the distal tip portion 234 engages the implant apparatus 290. Accordingly, the distal tip portion hardness can be greater than the first flex portion hardness of the first flex portion 240. As more specific examples, the distal tip portion hardness can correspond to a durometer value that is at least 40 D, at least 50 D, at least 60 D, at most 70 D, at most 55 D, at most 45 D, 40-55 D, 50-70 D, and/or 40-70 D.

Additionally, while it generally may be desirable to configure the distal region of the first shaft 204 to be relatively flexible, it also may be desirable to configure a portion of the first shaft 204 proximal to this distal region to be relatively stiff. For example, in regions in which the delivery shaft 202 does not bend significantly within the patient's vasculature, it may be desirable to configure corresponding regions of the first shaft 204 to be sufficiently stiff to resist deformation as the delivery shaft 202 is advanced through the guide catheter. Thus, the distal region bulk portion 260 can have a distal region bulk hardness that is greater than each of the first flex portion hardness, the second flex portion hardness, the third flex portion hardness, and the fourth flex portion hardness. As more specific examples, the distal region bulk hardness can correspond to a durometer value that is at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and/or 20-80 D.

While the present disclosure generally relates to examples in which the first flex portion 240, the second flex portion 244, the third flex portion 248, and the fourth flex portion 252 have distinct respective harnesses that are at least substantially uniform within each portion, it also is within the scope of the present disclosure that the hardness of the first shaft 204 can vary smoothly and/or continuously within the distal region 230. For example, the first shaft 204 can be formed of a material with a hardness that steadily increases from the first flex portion hardness within the first flex portion 240 to the fourth flex portion hardness within the fourth flex portion 252. Accordingly, in various examples, each of the first flex portion hardness, the second flex portion hardness, the third flex portion hardness, and/or the fourth flex portion hardness can refer to a maximum hardness, a minimum hardness, an average hardness, etc. characterizing the respective portion.

In some examples, and as shown in FIG. 9, the distal tip portion 234 includes a radiopaque marker 238 to assist the user in aligning the valve at the proper location on the balloon. The radiopaque marker 238 can include and/or be any of a variety of radiopaque materials, examples of which include tungsten, a tungsten extrusion, platinum, iridium a platinum iridium alloy, and/or a doped polymer material. Additionally, or alternatively, the distal tip portion 234 (and/or any other suitable portion of the first shaft 204) may include a radiopaque material to assist a user in locating a portion of the first shaft 204. For example, one or more layers and/or components of the first shaft 204 may include (e.g., be doped with) a radiopaque material such as barium sulfate and/or bismuth subcarbonate. In such examples, such a radiopaque material may be used in combination with or in lieu of the radiopaque marker 238.

In the present disclosure, the distal region 230 of the first shaft 204 generally corresponds to a distal-most portion of the first shaft 204 that includes the flex portions (e.g., the first flex portion 240, the second flex portion 244, the third flex portion 248, and the fourth flex portion 252) and at least a portion of the first shaft 204 extending proximally from these flex portions with more uniform and/or relatively high stiffness. The proximal region 270 of the first shaft 204 generally corresponds to a proximal-most portion of the first shaft 204, such as a portion that interfaces with and/or extends within the handle 201.

A boundary between the distal region 230 and the proximal region may be defined in any of a variety of manners. For example, and as shown in FIG. 9, the first shaft 204 can include a transition region 264 within which an outer diameter of the first shaft 204 varies between a first diameter 274 associated with the proximal region 270 and a second diameter 232 associated with the distal region 230, the second diameter 232 being greater than the first diameter 274. In some examples, the first diameter 274 also may be referred to as a proximal region outer diameter 274 and/or the second diameter 232 may be referred to as a distal region outer diameter 232. In some examples, and as shown in FIG. 9, the first shaft 204 has the distal region outer diameter 232 (e.g., a substantially constant diameter) throughout the length of the distal region 230 and/or fully to a distal end of the first shaft 204.

In some examples, the transition region 264 thus may separate the distal region 230 and the proximal region 270 such that the transition region 264 represents a boundary between the distal region 230 and the proximal region 270. In other examples, the distal region 230 or the proximal region 270 can include the transition region 264. For example, the proximal region 270 may be described as including a first proximal portion 272 and a second proximal portion 278 extending distal to the first proximal portion 272 between the first proximal portion 272 and the distal region 230. In some such examples, the transition region 264 can extend between and/or separate the first proximal portion 272 and the second proximal portion 278.

The first proximal portion 272 can be characterized by a first proximal portion outer diameter 274, while the second proximal portion 278 can be characterized by a second proximal portion outer diameter 280 that is greater than the first proximal portion outer diameter 274. In this manner, the first diameter 274 introduced above also can be referred to as the proximal region outer diameter 274 and/or as the first proximal region outer diameter 274. Similarly, the second diameter 232 introduced above also can be referred to as the distal region outer diameter 232 and/or as the second proximal portion outer diameter 280. Additionally or alternatively, in some examples, the second proximal portion outer diameter 280 is equal, or approximately equal, to the distal region outer diameter 232.

The distal region outer diameter 232 and/or the second proximal portion outer diameter 280 can assume any of a variety of values. As examples, the distal region outer diameter 232 and/or the second proximal portion outer diameter 280 can be at least 0.2 inches, at least 0.25 inches, at least 0.3 inches, at least 0.35 inches, at most 0.4 inches, at most 0.32 inches, at most 0.27 inches, at most 0.22 inches, 0.2-0.27 inches, 0.25-0.32 inches, 0.3-0.4 inches, 0.2-0.32 inches, 0.25-0.4 inches, and/or 0.2-0.4 inches. In particular, in some examples, the distal region outer diameter 232 is approximately equal to the shaft inner diameter 296 of the shaft 294 of the guide catheter as shown in FIG. 8.

Similarly, the proximal region outer diameter 274 can assume any of a variety of values. As examples, the proximal region outer diameter 274 can be at least 0.15 inches, at least 0.2 inches, at least 0.25 inches, at least 0.3 inches, at least 0.35 inches, at most 0.4 inches, at most 0.32 inches, at most 0.27 inches, at most 0.22 inches, at most 0.17 inches, 0.15-0.22 inches, 0.2-0.27 inches, 0.25-0.32 inches, 0.3-0.4 inches, 0.15-0.27 inches, 0.2-0.32 inches, 0.25-0.4 inches, 0.15-0.32 inches, 0.2-0.4 inches, and/or 0.15-0.4 inches.

In an example in which the proximal region 270 includes the first proximal portion 272 and the second proximal portion 278, the first proximal portion outer diameter 274 and the second proximal portion outer diameter 280 each can assume any of a variety of values. As examples, the first proximal portion outer diameter 274 can be at least 0.15 inches, at least 0.2 inches, at least 0.25 inches, at least 0.3 inches, at least 0.35 inches, at most 0.4 inches, at most 0.32 inches, at most 0.27 inches, at most 0.22 inches, at most 0.17 inches, 0.15-0.22 inches, 0.2-0.27 inches, 0.25-0.32 inches, 0.3-0.4 inches, 0.15-0.27 inches, 0.2-0.32 inches, 0.25-0.4 inches, 0.15-0.32 inches, 0.2-0.4 inches, and/or 0.15-0.4 inches. Additionally or alternatively, the second proximal portion outer diameter 280 can be at least 0.15 inches, at least 0.2 inches, at least 0.25 inches, at least 0.3 inches, at least 0.35 inches, at most 0.4 inches, at most 0.32 inches, at most 0.27 inches, at most 0.22 inches, at most 0.17 inches, 0.15-0.22 inches, 0.2-0.27 inches, 0.25-0.32 inches, 0.3-0.4 inches, 0.15-0.27 inches, 0.2-0.32 inches, 0.25-0.4 inches, 0.15-0.32 inches, 0.2-0.4 inches, and/or 0.15-0.4 inches.

Each portion and/or region of the first shaft 204 can have any suitable linear dimension, e.g., as measured along a direction parallel to the longitudinal axis 203. For example, the distal tip portion 234 can have a distal tip portion length 236 that is at least 0.01 inches, at least 0.05 inches, at least 0.1 inches, at least 0.5 inches, at most 1 inch, at most 0.2 inches, at most 0.07 inches, at most 0.02 inches, 0.01-0.07 inches, 0.05-0.2 inches, 0.1-1 inches, 0.01-0.2 inches, 0.05-1 inches, and/or 0.01-1 inches.

Additionally or alternatively, the first flex portion 240 can have a first flex portion length 242 that is at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and/or 0.5-6 inches.

Additionally or alternatively, the second flex portion 244 can have a second flex portion length 246 that is at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and/or 0.5-6 inches.

Additionally or alternatively, when present, the third flex portion 248 can have a third flex portion length 250 that is at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and/or 0.5-6 inches.

Additionally or alternatively, when present, the fourth flex portion 252 can have a fourth flex portion length 254 that is at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and/or 0.5-6 inches.

In some examples, a length (e.g., a total length) of the flex portions of the distal region 230 can correspond to a length of a flex region of the shaft 294 of the guide catheter through which the first shaft 204 extends. For example, the sum of the distal tip portion length 236, the first flex portion length 242, the second flex portion length 246, the third flex portion length 250 (when present), and the fourth flex portion length 254 (when present) can be equal, or approximately equal, to a length of a distal end region of the shaft 294 that is configured to flex in a region that extends across the atrial septum and into coaxiality with the native mitral valve annulus during a mitral valve replacement procedure. By contrast, a remainder of the first shaft 204 (e.g., the distal region bulk portion 260 and/or the proximal region 270 can extend through a portion of the shaft 294 that need not exhibit such bending during a mitral valve replacement procedure, such that such regions of the first shaft 204 and/or of the shaft 294 can exhibit the relatively high material hardness values disclosed herein.

Additionally or alternatively, the distal region bulk portion 260 can have a distal region bulk portion length 262 that is at least 20 inches, at least 25 inches, at least 30 inches, at least 35 inches, at least 40 inches, at most 50 inches, at most 37 inches, at most 32 inches, at most 27 inches, at most 22 inches, 20-27 inches, 25-32 inches, 30-37 inches, 35-50 inches, 20-32 inches, 25-37 inches, 30-50 inches, 20-37 inches, 25-50 inches, and/or 20-50 inches. As shown in FIG. 9, the distal region bulk portion length 262 can extend to the second proximal portion 278 (solid arrow) or to the transition region 264 (dashed arrow).

The first shaft 204 also may have any suitable length between the proximal end thereof and the transition region 264, which in some examples may be referred to as a proximal portion length 276 of the proximal region 270 and/or as a first proximal portion length 276 of the first proximal portion 272. As examples, the proximal region length 276 can be at least 2 inches, at least 3 inches, at least 4 inches, at least 5 inches, at most 6 inches, at most 4.5 inches, at most 3.5 inches, at most 2.5 inches, 2-3.5 inches, 3-4.5 inches, 4-6 inches, 2-4.5 inches, 3-6 inches, and/or 2-6 inches.

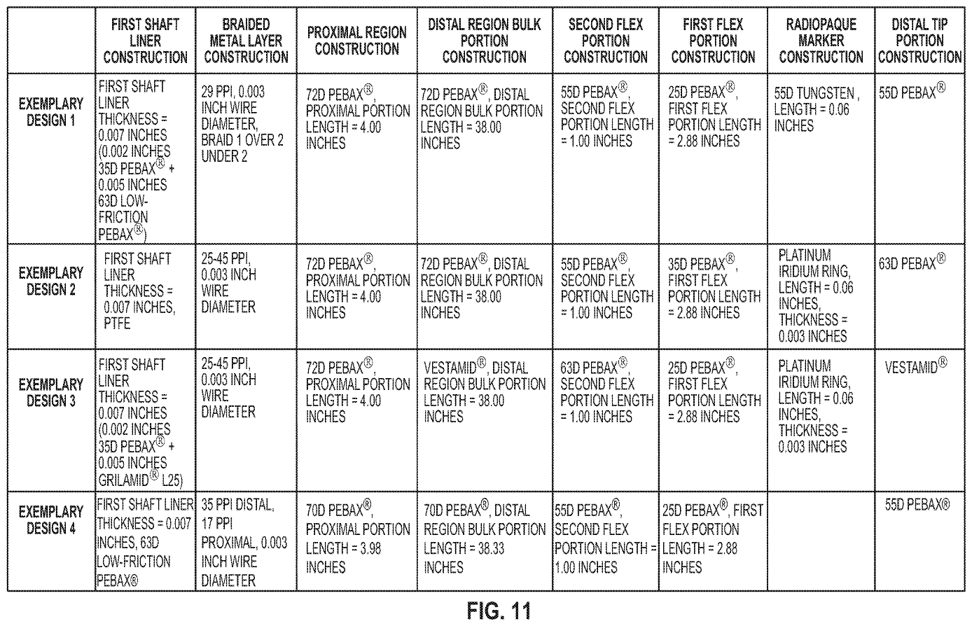

The first shaft 204 also may be described in terms of a layer-wise construction thereof. Such layer-wise construction further may be understood with reference to FIG. 10, which represents a cross-sectional view of the first shaft 204 as viewed along the line 10 -10 in FIG. 9.

As shown in FIGS. 9-10, the first shaft 204 includes a first shaft lumen 216 configured to receive the second shaft 206 therein (as depicted in FIG. 8). In some examples, and as shown in FIGS. 9-10, the first shaft 204 includes a first shaft liner 212 that at least partially defines the first shaft lumen 216. The first shaft 204 further can include a first shaft body 210 surrounding the first shaft liner 212 and defining an external surface of the first shaft 204.

The first shaft liner 212 can extend along any suitable length of the first shaft 204. For example, the first shaft liner 212 may extend fully to a distal end of the first shaft 204 or may terminate short of the distal end of the first shaft 204. Similarly, the first shaft liner 212 may extend fully to a proximal end of the first shaft 204 or may terminate short of the proximal end of the first shaft 204.

The first shaft 204 and/or the first shaft body 210 can be formed of any of a variety of materials. For example, the first shaft liner 212 and/or the first shaft body 210 can include a polyether block amide, such as that known as Pebax®. Additionally or alternatively, in some examples portions of the first shaft 204 and/or of the first shaft body 210 can be formed of a polyamide such as that known as Vestamid®. In particular, in various examples, Vestamid® can represent a strong and stiff alternative to high-durometer Pebax® material constructions.

The first shaft lumen 216 can have any of a variety of dimensions, such as may correspond to dimensions of at least a portion of the second shaft 206. For example, and as shown in FIGS. 9-10, the first shaft lumen 216 can have a lumen inner diameter 218 that is at least 0.10 inches, at least 0.15 inches, at least 0.20 inches, at least 0.25 inches, at least 0.30 inches, at most 0.35 inches, at most 0.27 inches, at most 0.22 inches, at most 0.17 inches, at most 0.12 inches, 0.10-0.17 inches, 0.15-0.22 inches, 0.20-0.27 inches, 0.25-0.35 inches, 0.10-0.22 inches, 0.15-0.27 inches, 0.20-0.35 inches, 0.10-0.27 inches, 0.15-0.35 inches, and/or 0.10-0.35 inches.

In some examples, the first shaft lumen 216 has an inner diameter that varies along a length of the first shaft 204, such as to produce a region of a relatively large inner diameter at a distal end of the first shaft lumen 216. For example, and as shown in FIG. 9, the first shaft lumen 216 can have the lumen inner diameter 218 in at least a portion of the distal region 230 and can have a lumen distal inner diameter 220 in at least a portion of the distal tip portion 234. In such examples, the lumen inner diameter 218 also may be referred to as a lumen proximal inner diameter 218.

In the example of FIG. 9, the first shaft lumen 216 has the lumen distal inner diameter 220 in a region that encompasses a full length of the distal tip portion 234 and a partial length of the first flex portion 240. In other examples, the first shaft lumen 216 can have the lumen distal inner diameter 220 in a region that encompasses a full length of each of the distal tip portion 234 and the first flex portion 240, or that encompasses only a portion of a length of the distal tip portion 234.

In the example of FIG. 9, the lumen distal inner diameter 220 is greater than the lumen proximal inner diameter 218. Such a configuration can facilitate receiving at least a portion of the implant apparatus 290 (FIG. 8) within the distal tip portion 234, such as a formed proximal section of an inflatable balloon. For example, such a configuration can facilitate mating the distal tip portion 234 of the first shaft 204 with the implant apparatus 290. Configuring a portion of a remainder of the first shaft 204 to have the relatively small lumen proximal inner diameter 218 can facilitate tracking with the second shaft 206 through such portions of the delivery apparatus 200. In some examples, the first shaft lumen 216 has the lumen proximal inner diameter 218 in at least a portion of the proximal region 270 as well.

The lumen proximal inner diameter 218 and the lumen distal inner diameter 220 can have any suitable values. As examples, the lumen proximal inner diameter 218 can be at least 0.10 inches, at least 0.15 inches, at least 0.20 inches, at least 0.25 inches, at least 0.30 inches, at most 0.35 inches, at most 0.27 inches, at most 0.22 inches, at most 0.17 inches, at most 0.12 inches, 0.10-0.17 inches, 0.15-0.22 inches, 0.20-0.27 inches, 0.25-0.35 inches, 0.10-0.22 inches, 0.15-0.27 inches, 0.20-0.35 inches, 0.10-0.27 inches, 0.15-0.35 inches, and/or 0.10-0.35 inches. Additionally or alternatively, the lumen distal inner diameter 220 can be at least 0.10 inches, at least 0.15 inches, at least 0.20 inches, at least 0.25 inches, at least 0.30 inches, at most 0.35 inches, at most 0.27 inches, at most 0.22 inches, at most 0.17 inches, at most 0.12 inches, 0.10-0.17 inches, 0.15-0.22 inches, 0.20-0.27 inches, 0.25-0.35 inches, 0.10-0.22 inches, 0.15-0.27 inches, 0.20-0.35 inches, 0.10-0.27 inches, 0.15-0.35 inches, and/or 0.10-0.35 inches.

The first shaft lumen 216 can transition from the lumen proximal inner diameter 218 to the lumen distal inner diameter 220 in any suitable manner. For example, and as shown in FIG. 9, the first shaft 204 and/or the first shaft liner 212 can include and/or define a lumen shoulder 222 at which the inner diameter of the first shaft lumen 216 changes. Specifically, the first shaft lumen 216 can have the lumen proximal inner diameter 218 on a proximal side of the lumen shoulder 222 and can have the lumen distal inner diameter 220 on a distal side of the lumen shoulder 222. In some examples, and as shown in FIG. 9, the lumen shoulder 222 can represent a region in which the inner diameter of the first shaft lumen 216 abruptly changes from the lumen proximal inner diameter 218 to the lumen distal inner diameter 220. In other examples, the lumen shoulder 222 can include and/or be a ramped surface corresponding to a more gradual variation in this inner diameter. For example, the lumen shoulder 222 can include and/or be a ramped surface that extends over a length (as measured along a direction parallel to the longitudinal axis 203) that is approximately 0.5 inches.

In other examples, the first shaft lumen 216 can have a consistent inner diameter (e.g., the lumen inner diameter 218) across a full length of the first shaft lumen 216 and/or within the distal tip portion 234.

The first shaft liner 212 can be formed of any of a variety of materials. For example, the first shaft liner 212 can include a polyether block amide, such as that known as Pebax®. As another example, the first shaft liner 212 can include and/or be a low-friction material such as polytetrafluoroethylene (PTFE) and/or a polyamide material such as that known as Grilamid®. For example, the use of PTFE can provide for a more lubricious surface for interaction with a portion of the implant apparatus 290, such as a portion of an inflatable balloon.

In some examples, the first shaft liner 212 can include a first liner material and a second liner material with a hardness that is greater than that of the first liner material. In particular, in some examples, the first liner material includes and/or is Pebax®, and the second liner material includes Pebax® in combination with a low-friction compound such as that known as Foster ProPell™. In some such examples, the first liner material is thinner than the second liner material.

The first shaft liner 212 can have any suitable thickness. As examples, the first shaft liner 212 can have a first shaft liner thickness 214 that is at least 0.002 inches, at least 0.005 inches, at least 0.01 inches, at most 0.012 inches, at most 0.007 inches, at most 0.003 inches, 0.002-0.007 inches, 0.005-0.012 inches, and/or 0.002-0.012 inches.

The first shaft body 210 also can have any of a variety of material constructions and/or properties. For example, the first shaft body 210 can form a portion of each of the first flex portion 240, the second flex portion 244, the third flex portion 248, the fourth flex portion 252, the distal region bulk portion 260, and/or the proximal region 270 (as applicable). Accordingly, the first shaft body 210 can have the first flex portion hardness within the first flex portion 240, can have the second flex portion hardness within the second flex portion 244, can have the third flex portion hardness within the third flex portion 248, can have the fourth flex portion hardness within the fourth flex portion 252, and/or can have the distal region bulk hardness within the distal region bulk portion 260. Similarly, the first shaft body 210 can form at least a portion of the distal tip portion 234 and/or can have the distal tip portion hardness within the distal tip portion 234.

As shown in FIG. 9, the first shaft 204 additionally can include a braided metal layer 224 surrounding at least a portion of the first shaft liner 212. The braided metal layer 224 can contribute to the stiffness and/or pushability of the first shaft 204. The braided metal layer 224 can be formed from braided metal wire wound around the first shaft liner 212, and the first shaft body 210 can surround and encapsulate the braided metal layer 224. In particular examples, the first shaft 204 can be formed by forming the first shaft liner 212, wrapping metal wire around the first shaft liner 212 to form the braided metal layer 224, placing a polymeric sleeve over the braided layer, and reflowing the sleeve to form the first shaft body 210 as a uniform laminate layer surrounding the liner. The first shaft body 210 can be formed of any suitable material, such as a thermoplastic elastomer and/or Pebax®.

The braided metal layer 224 can be constructed from metal wire (e.g., stainless steel wire) in any of a variety of configurations. As examples, the metal wires forming the braided metal layer 224 can have a diameter that is at least 0.002 inches, at least 0.003 inches, at least 0.004 inches, at least 0.005 inches, and at most 0.006 inches. Additionally or alternatively, the metal wires can be braided with a pick density that is at least 10 picks per inch (ppi), at least 20 ppi, at least 30 ppi, at least 40 ppi, at least 50 ppi, at most 60 ppi, at most 45 ppi, at most 35 ppi, at most 25 ppi, at most 15 ppi, 10-25 ppi, 20-35 ppi, 30-45 ppi, 40-60 ppi, 10-35 ppi, 20-45 ppi, 30-60 ppi, 10-45 ppi, and/or 20-60 ppi. In some cases, a determination of the pick density of the braided metal layer 224 is at least partially based on a tradeoff between flexural rigidity and torsional rigidity. For example, within certain pick density ranges, increasing the pick density corresponds to an increase in flexural rigidity and a decrease in torsional rigidity. Thus, the pick density of the braided metal layer 224 may be selected to correspond to a value that maintains both flexural rigidity and torsional rigidity within acceptable parameters.

In some examples, the pick density of the braided metal layer 224 may vary along a length of the braided metal layer 224. For example, the pick density of the braided metal layer 224 may be greater within the distal region 230 than in the proximal region 270. As a more specific example, the pick density of the braided metal layer 224 within at least a portion of the distal region 230 may be 30-45 ppi and the pick density of the braided metal layer 224 within at least a portion of the proximal region 270 may be 10-25 ppi. In some such examples, the braided metal layer 224 can be a continuous layer that exhibits different pick densities in different regions of the braided metal layer 224. Additionally, or alternatively, the braided metal layer 224 can include multiple separate braided segments with different pick densities arranged adjacent to one another to collectively form at least a portion of the braided metal layer 224.

In some examples, the braided metal layer 224 extends along only a portion of a full length of the first shaft 204. For example, and as shown in FIG. 9, the braided metal layer 224 can terminate short of a proximal end of the first shaft 204 by a braided layer terminal offset 226 that is at least 0.1 inches, at least 0.5 inches, at least 1 inch, at least 2 inches, at least 3 inches, at least 4 inches, at most 5 inches, at most 3.5 inches, at most 2.5 inches, at most 1.5 inches, at most 0.75 inches, at most 0.3 inches, 0.1-0.75 inches, 0.5-1.5 inches, 1-2.5 inches, 2-3.5 inches, 3-5 inches, 0.1-1.5 inches, 0.5-2.5 inches, 1-3.5 inches, 2-5 inches, 0.1-2.5 inches, 0.5-3.5 inches, 1-5 inches, 0.1-2.5 inches, 0.5-3.5 inches, 1-5 inches, 0.1-3.5 inches 0.5-5 inches, and/or 0.1-5 inches. In other examples, the braided metal layer 224 can extend fully to the proximal end of the first shaft 204.

Additionally or alternatively, and as shown in FIG. 9, the braided metal layer 224 can extend partially within and/or terminate within the distal tip portion 234, such as within a region within which the radiopaque marker 238 extends. As a more specific example, the braided metal layer 224 may terminate under the radiopaque marker 238. In other examples, the braided metal layer 224 can terminate proximally of the distal tip portion 234, such as within the first flex portion 240.

FIG. 11 is a table representing various construction features of each of three exemplary designs of the first shaft 204. Each design represented in FIG. 11 corresponds to an example in which the distal region 230 includes the first flex portion 240 and the second flex portion 244, with the distal region bulk portion 260 extending between and interconnecting the second flex portion 244 and the transition region 264. In each of these examples, the transition region 264 separates the distal region 230 and the proximal region 270.

As shown in FIG. 11, the first shaft liner 212 of the first shaft 204 of Exemplary Design 1 includes a first liner material in the form of a 0.002-inch-thick layer of Pebax® with a hardness of 35 D. The first shaft liner 212 in this example additionally includes a second liner material in the form of a 0.005-inch-thick layer of Pebax® in combination with Foster ProPell™. In this example, the braided metal layer 224 is formed of metal wires with a diameter of 0.003 inches braided with a pick density of 29 PPI. The distal tip portion 234 is formed of Pebax® with a hardness of 55 D, and the radiopaque marker 238 is a tungsten extrusion with a hardness of 55 D and a length of 0.06 inches. The first flex portion 240 in this example is formed of Pebax® with a hardness of 25 D and has a first flex portion length 242 of 2.88 inches. The second flex portion 244 in this example is formed of Pebax® with a hardness of 55 D and a second flex portion length 246 of 1.00 inches. In this example, the distal region bulk portion 260 is formed of Pebax® with a hardness of 72 D and has a distal region bulk portion length 262 of 38.00 inches. The proximal region 270 is formed of Pebax® with a hardness of 72 D and has a proximal portion length 276 of 4.00 inches.

The first shaft liner 212 of the first shaft 204 of Exemplary Design 2 is a 0.007-inch-thick layer of PTFE. In this example, the braided metal layer 224 is formed of metal wires with a diameter of 0.003 inches braided with a pick density of 25-45 PPI. The distal tip portion 234 is formed of Pebax® with a hardness of 63 D, and the radiopaque marker 238 is a platinum iridium ring with a length of 0.06 inches and a thickness of 0.003 inches. The first flex portion 240 in this example is formed of Pebax® with a hardness of 35 D and has a first flex portion length 242 of 2.88 inches. The second flex portion 244 in this example is formed of Pebax® with a hardness of 55 D and a second flex portion length 246 of 1.00 inches. In this example, the distal region bulk portion 260 is formed of Pebax® with a hardness of 72 D and has a distal region bulk portion length 262 of 38.00 inches. The proximal region 270 is formed of Pebax® with a hardness of 72 D and has a proximal portion length 276 of 4.00 inches.

The first shaft liner 212 of the first shaft 204 of Exemplary Design 3 includes a first liner material in the form of a 0.002-inch-thick layer of Pebax® with a hardness of 35 D. The first shaft liner 212 in this example additionally includes a second liner material in the form of a 0.005-inch-thick layer of Grilamid® L25. In this example, the braided metal layer 224 is formed of metal wires with a diameter of 0.003 inches braided with a pick density of 25-45 PPI. The distal tip portion 234 is formed of Vestamid®, and the radiopaque marker 238 is a platinum iridium ring with a length of 0.06 inches and a thickness of 0.003 inches. The first flex portion 240 in this example is formed of Pebax® with a hardness of 25 D and has a first flex portion length 242 of 2.88 inches. The second flex portion 244 in this example is formed of Pebax® with a hardness of 63 D and a second flex portion length 246 of 1.00 inches. In this example, the distal region bulk portion 260 is formed of Pebax® with a hardness of 72 D and has a distal region bulk portion length 262 of 38.00 inches. The proximal region 270 is formed of Pebax® with a hardness of 72 D and has a proximal portion length 276 of 4.00 inches.

The first shaft liner 212 of the first shaft 204 of Exemplary Design 4 is a 0.007-inch-thick layer of Pebax® in combination with Foster ProPell™ and has a hardness of 63 D. In this example, the braided metal layer 224 is formed of metal wires with a diameter of 0.003 inches braided with a pick density of 35 ppi within at least a portion of the distal region 230 and 17 ppi within at least a portion of the proximal region 270. The braided metal layer 224 extends between the proximal end of the first shaft 204 and a location that is 0.06 inches short of the distal end of the first shaft 204. The distal tip portion 234 is formed of Pebax® with a hardness of 55 D. The first flex portion 240 in this example is formed of Pebax® with a hardness 25 D and has a first flex portion length 242 of 2.88 inches. The second flex portion 244 in this example is formed of Pebax® with a hardness of 55 D and has a second flex portion length 246 of 1.00 inches. In this example, the distal region bulk portion 260 is formed of Pebax® with a hardness of 70 D and has a distal region bulk portion length 262 of 38.33 inches. The proximal region 270 is formed of Pebax® with a hardness of 70 D and has a proximal portion length 276 of 3.98 inches.

In Exemplary Design 4, the first shaft liner 212, the first flex portion 240, the second flex portion 244, and the distal region bulk portion 262 include barium sulfate and the distal tip portion 234 includes bismuth subcarbonate. In this example, the first shaft 204 does not include a radiopaque marker 238. It is to be understood, however, that any of the features of Exemplary Design 4 could be used in an example that does include a radiopaque marker 238. For example, another example of a first shaft 204 according to the present disclosure may be identical to Exemplary Design 4 aside from the inclusion of the radiopaque marker 238 in any suitable form.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

### Example 1

A delivery apparatus comprising: a delivery shaft comprising: a first shaft comprising a distal region and a proximal region; and a second shaft disposed radially interiorly of the first shaft, wherein the first shaft is configured to translate relative to the second shaft along a longitudinal axis of the delivery shaft, wherein the second shaft comprises a distal end region configured to support a prosthetic implant thereon, and wherein the distal region of the first shaft comprises: a distal tip portion configured to engage an implant apparatus supported by the second shaft and having a distal tip portion hardness; a first flex portion extending on a proximal side of the distal tip portion and having a first flex portion hardness that is less than the distal tip portion hardness; a second flex portion extending on a proximal side of the first flex portion and having a second flex portion hardness that is greater than the first flex portion hardness; and a distal region bulk portion extending between the second flex portion and the proximal region and having a distal region bulk hardness that is greater than the second flex portion hardness.

### Example 2

The delivery apparatus of any example herein, particularly Example 1, wherein the implant apparatus comprises a portion of the prosthetic implant.

### Example 3

The delivery apparatus of any example herein, particularly any one of Examples 1-2, further comprising an inflatable balloon mounted on the distal end region of the second shaft, and wherein the implant apparatus comprises a portion of the inflatable balloon.

### Example 4

The delivery apparatus of any example herein, particularly any one of Examples 1-3, wherein the delivery shaft is configured to extend into a patient's vasculature to transport a prosthetic device toward a target implantation site.

### Example 5

The delivery apparatus of any example herein, particularly any one of Examples 1-4, further comprising a handle configured to facilitate translation of the first shaft relative to the second shaft, and wherein the delivery shaft extends distally from the handle.

### Example 6

The delivery apparatus of any example herein, particularly Example 5, wherein the handle comprises one or more articulation members configured to be adjusted by a user to articulate a distal end portion of the delivery shaft.

### Example 7

The delivery apparatus of any example herein, particularly Example 5, wherein the handle is free of articulation members for bending a distal end portion of the delivery shaft.

### Example 8

The delivery apparatus of any example herein, particularly any one of Examples 1-7, wherein the distal tip portion has a distal tip portion length that is one or more of at least 0.01 inches, at least 0.05 inches, at least 0.1 inches, at least 0.5 inches, at most 1 inch, at most 0.2 inches, at most 0.07 inches, at most 0.02 inches, 0.01-0.07 inches, 0.05-0.2 inches, 0.1-1 inches, 0.01-0.2 inches, 0.05-1 inches, and 0.01-1 inches.

### Example 9

The delivery apparatus of any example herein, particularly any one of Examples 1-8, wherein the distal tip portion comprises a radiopaque marker.

### Example 10

The delivery apparatus of any example herein, particularly Example 9, wherein the radiopaque marker comprises one or more of tungsten, platinum, iridium, a platinum iridium alloy, and a doped polymer material.

### Example 11

The delivery apparatus of any example herein, particularly any one of Examples 1-10, wherein the distal tip portion hardness corresponds to a durometer value that is one or more of at least 40 D, at least 50 D, at least 60 D, at most 70D, at most 55D, at most 45 D, 40-55 D, 50-70 D, and 40-70 D.

### Example 12

The delivery apparatus of any example herein, particularly any one of Examples 1-11, wherein the first flex portion has a first flex portion length that is one or more of at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and 0.5-6 inches.

### Example 13

The delivery apparatus of any example herein, particularly any one of Examples 1-12, wherein the first flex portion hardness corresponds to a durometer value that is one or more of at least 10 D, at least 20 D, at least 30 D, at least 40 D, at most 50 D, at most 35 D, at most 25 D, at most 15 D, 10-25D, 20-35 D, 30-50 D, 10-35 D, 20-50 D, and 10-50 D.

### Example 14

The delivery apparatus of any example herein, particularly any one of Examples 1-13, wherein the second flex portion has a second flex portion length that is one or more of at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and 0.5-6 inches.

### Example 15

The delivery apparatus of any example herein, particularly any one of Examples 1-14, wherein the second flex portion hardness corresponds to a durometer value that is one or more of at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and 20-80 D.

### Example 16

The delivery apparatus of any example herein, particularly any one of Examples 1-15, wherein the distal region bulk portion has a distal region bulk portion length that is one or more of at least 20 inches, at least 25 inches, at least 30 inches, at least 35 inches, at least 40 inches, at most 50 inches, at most 37 inches, at most 32 inches, at most 27 inches, at most 22 inches, 20-27 inches, 25-32 inches, 30-37 inches, 35-50 inches, 20-32 inches, 25-37 inches, 30-50 inches, 20-37 inches, 25-50 inches, and 20-50 inches.

### Example 17

The delivery apparatus of any example herein, particularly any one of Examples 1-16, wherein the distal region bulk hardness corresponds to a durometer value that is one or more of at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and 20-80 D.

### Example 18

The delivery apparatus of any example herein, particularly any one of Examples 1-17, wherein the distal region has a distal region outer diameter that is one or more of at least 0.2 inches, at least 0.25 inches, at least 0.3 inches, at least 0.35 inches, at most 0.4 inches, at most 0.32 inches, at most 0.27 inches, at most 0.22 inches, 0.2-0.27 inches, 0.25-0.32 inches, 0.3-0.4 inches, 0.2-0.32 inches, 0.25-0.4 inches, and 0.2-0.4 inches.

### Example 19

The delivery apparatus of any example herein, particularly any one of Examples 1-18, wherein the distal tip portion hardness corresponds to a durometer value that is 40-70 D, wherein the first flex portion hardness corresponds to a durometer value that is 20-50 D, wherein the second flex portion hardness corresponds to a durometer value that is 40-80 D, and wherein the distal region bulk hardness corresponds to a durometer value that is 60-80 D.

### Example 20

The delivery apparatus of any example herein, particularly any one of Examples 1-19, wherein the distal tip portion hardness corresponds to a durometer value that is 50-60 D, wherein the first flex portion hardness corresponds to a durometer value that is 20-35 D, wherein the second flex portion hardness corresponds to a durometer value that is 50-65 D, and wherein the distal region bulk hardness corresponds to a durometer value that is 65-80 D.

### Example 21

The delivery apparatus of any example herein, particularly any one of Examples 1-20, wherein the proximal region has a proximal region outer diameter that is smaller than a distal region outer diameter of the distal region.

### Example 22

The delivery apparatus of any example herein, particularly any one of Examples 1-21, wherein the proximal region comprises a first proximal portion and a second proximal portion extending distal to the first proximal portion between the first proximal portion and the distal region, and wherein the first proximal portion has a first proximal portion outer diameter that is less than the distal region outer diameter.

### Example 23

The delivery apparatus of any example herein, particularly Example 22, wherein the second proximal portion has a second proximal portion outer diameter that is equal, or approximately equal, to the distal region outer diameter.

### Example 24

The delivery apparatus of any example herein, particularly any one of Examples 22-23, wherein the first proximal portion has a first proximal portion length that is one or more of at least 2 inches, at least 3 inches, at least 4 inches, at least 5 inches, at most 6 inches, at most 4.5 inches, at most 3.5 inches, at most 2.5 inches, 2-3.5 inches, 3-4.5 inches, 4-6 inches, 2-4.5 inches, 3-6 inches, and 2-6 inches.

### Example 25

The delivery apparatus of any example herein, particularly any one of Examples 1-24, wherein the proximal region has a proximal region outer diameter that is one or more of at least 0.15 inches, at least 0.2 inches, at least 0.25 inches, at least 0.3 inches, at least 0.35 inches, at most 0.4 inches, at most 0.32 inches, at most 0.27 inches, at most 0.22 inches, at most 0.17 inches, 0.15-0.22 inches, 0.2-0.27 inches, 0.25-0.32 inches, 0.3-0.4 inches, 0.15-0.27 inches, 0.2-0.32 inches, 0.25-0.4 inches, 0.15-0.32 inches, 0.2-0.4 inches, and 0.15-0.4 inches.

### Example 26

The delivery apparatus of any example herein, particularly any one of Examples 1-25, wherein the proximal region comprises a first proximal portion and a second proximal portion extending distal to the first proximal portion between the first proximal portion and the distal region, wherein the first proximal portion has a first proximal portion outer diameter, and wherein the second proximal portion has a second proximal portion outer diameter that is greater than the first proximal portion outer diameter.

### Example 27

The delivery apparatus of any example herein, particularly Example 26, wherein the first proximal portion outer diameter is one or more of at least 0.15 inches, at least 0.2 inches, at least 0.25 inches, at least 0.3 inches, at least 0.35 inches, at most 0.4 inches, at most 0.32 inches, at most 0.27 inches, at most 0.22 inches, at most 0.17 inches, 0.15-0.22 inches, 0.2-0.27 inches, 0.25-0.32 inches, 0.3-0.4 inches, 0.15-0.27 inches, 0.2-0.32 inches, 0.25-0.4 inches, 0.15-0.32 inches, 0.2-0.4 inches, and 0.15-0.4 inches.

### Example 28

The delivery apparatus of any example herein, particularly any one of Examples 26-27, wherein the second proximal portion outer diameter is one or more of at least 0.15 inches, at least 0.2 inches, at least 0.25 inches, at least 0.3 inches, at least 0.35 inches, at most 0.4 inches, at most 0.32 inches, at most 0.27 inches, at most 0.22 inches, at most 0.17 inches, 0.15-0.22 inches, 0.2-0.27 inches, 0.25-0.32 inches, 0.3-0.4 inches, 0.15-0.27 inches, 0.2-0.32 inches, 0.25-0.4 inches, 0.15-0.32 inches, 0.2-0.4 inches, and 0.15-0.4 inches.

### Example 29

The delivery apparatus of any example herein, particularly any one of Examples 1-28, wherein the first shaft comprises a transition region in which an outer diameter of the first shaft varies between a first diameter and a second diameter that is greater than the first diameter.

### Example 30

The delivery apparatus of any example herein, particularly Example 29, wherein the transition region separates the distal region and the proximal region.

### Example 31

The delivery apparatus of any example herein, particularly Example 29, wherein the proximal region comprises the transition region.

### Example 32

The delivery apparatus of any example herein, particularly Example 31, wherein the proximal region comprises a first proximal portion and a second proximal portion, and wherein the transition region extends between the first proximal portion and the second proximal portion.

### Example 33

The delivery apparatus of any example herein, particularly any one of Examples 1-32, wherein the distal region of the first shaft further comprises a third flex portion extending on a proximal side of the second flex portion and having a third flex portion hardness that is greater than the second flex portion hardness.

### Example 34

The delivery apparatus of any example herein, particularly Example 33, wherein the first shaft comprises a first shaft liner that at least partially defines a first shaft lumen and a first shaft body surrounding the first shaft liner and defining an external surface of the first shaft, wherein the first shaft body forms a portion of the third flex portion, and wherein the first shaft body has the third flex portion hardness within the third flex portion.

### Example 35

The delivery apparatus of any example herein, particularly any one of Examples 33-34, wherein the distal region bulk portion extends between the third flex portion and the proximal region, and wherein the distal region bulk hardness is greater than the third flex portion hardness.

### Example 36

The delivery apparatus of any example herein, particularly any one of Examples 33-35, wherein the third flex portion has a third flex portion length that is one or more of at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and 0.5-6 inches.

### Example 37

The delivery apparatus of any example herein, particularly any one of Examples 33-36, wherein the third flex portion hardness corresponds to a durometer value that is one or more of at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and 20-80 D.

### Example 38

The delivery apparatus of any example herein, particularly any one of Examples 33-37, wherein the distal region of the first shaft further comprises a fourth flex portion extending on a proximal side of the third flex portion and having a fourth flex portion hardness that is greater than the third flex portion hardness.

### Example 39

The delivery apparatus of any example herein, particularly Example 38, wherein the first shaft comprises a first shaft liner that at least partially defines a first shaft lumen and a first shaft body surrounding the first shaft liner and defining an external surface of the first shaft, wherein the first shaft body forms a portion of the fourth flex portion, and wherein the first shaft body has the third flex portion hardness within the fourth flex portion.

### Example 40

The delivery apparatus of any example herein, particularly any one of Examples 38-39, wherein the distal region bulk portion extends between the fourth flex portion and the proximal region, and wherein the distal region bulk hardness is greater than the fourth flex portion hardness.

### Example 41

The delivery apparatus of any example herein, particularly any one of Examples 38-40, wherein the fourth flex portion has a fourth flex portion length that is one or more of at least 0.5 inches, at least 1 inch, at least 2 inches, at least 3 inches, at least 4 inches, at least 5 inches, at most 6 inches, at most 4.5 inches, at most 3.5 inches, at most 2.5 inches, at most 1.5 inches, at most 0.75 inches, 0.5-1.5 inches, 1-2.5 inches, 2-3.5 inches, 3-4.5 inches, 4-6 inches, 0.5-2.5 inches, 1-3.5 inches, 2-4.5 inches, 3-6 inches, 0.5-3.5 inches, 1-4.5 inches, 2-6 inches, 0.5-4.5 inches, 1-6 inches, and 0.5-6 inches.

### Example 42

The delivery apparatus of any example herein, particularly any one of Examples 38-41, wherein the fourth flex portion hardness corresponds to a durometer value that is one or more of at least 20 D, at least 30 D, at least 40 D, at least 50 D, at least 60 D, at least 70 D, at most 80 D, at most 65 D, at most 55 D, at most 45 D, at most 35 D, at most 25 D, 20-35 D, 30-45 D, 40-55 D, 50-65 D, 60-80 D, 20-45 D, 30-65 D, 40-80 D, 20-65 D, 30-80 D, and 20-80 D.

### Example 43

The delivery apparatus of any example herein, particularly any one of Examples 38-42, wherein the distal tip portion hardness corresponds to a durometer value that is 40-70 D, wherein the first flex portion hardness corresponds to a durometer value that is 20-40 D, wherein the second flex portion hardness corresponds to a durometer value that is 20-45 D, wherein the third flex portion hardness corresponds to a durometer value that is 30-65 D, wherein the fourth flex portion hardness corresponds to a durometer value that is 40-80 D, and wherein the distal region bulk hardness corresponds to a durometer value that is 60-80 D.

### Example 44

The delivery apparatus of any example herein, particularly any one of Examples 38-43, wherein the distal tip portion hardness corresponds to a durometer value that is 50-60 D, wherein the first flex portion hardness corresponds to a durometer value that is 20-30 D, wherein the second flex portion hardness corresponds to a durometer value that is 30-40 D, wherein the third flex portion hardness corresponds to a durometer value that is 45-60 D, wherein the fourth flex portion hardness corresponds to a durometer value that is 50-70 D, and wherein the distal region bulk hardness corresponds to a durometer value that is 65-80 D.

### Example 45

The delivery apparatus of any example herein, particularly any one of Examples 1-44, wherein the first shaft comprises a first shaft lumen that receives the second shaft therein.

### Example 46

The delivery apparatus of any example herein, particularly Example 45, wherein the first shaft comprises a first shaft liner that at least partially defines the first shaft lumen.

### Example 47

The delivery apparatus of any example herein, particularly Example 46, wherein the first shaft liner comprises a thermoplastic elastomer formed from polyether block amides.

### Example 48

The delivery apparatus of any example herein, particularly any one of Examples 46-47, wherein the first shaft liner comprises a first liner material and a second liner material with a hardness that is greater than that of the first liner material.

### Example 49

The delivery apparatus of any example herein, particularly Example 48, wherein the first liner material is thinner than the second liner material.

### Example 50

The delivery apparatus of any example herein, particularly any one of Examples 46-49, wherein the first shaft liner has a first shaft liner thickness that is one or more of at least 0.002 inches, at least 0.005 inches, at least 0.01 inches, at most 0.012 inches, at most 0.007 inches, at most 0.003 inches, 0.002-0.007 inches, 0.005-0.012 inches, and 0.002-0.012 inches.

### Example 51

The delivery apparatus of any example herein, particularly any one of Examples 46-50, wherein the first shaft comprises a braided metal layer surrounding the first shaft liner.

### Example 52

The delivery apparatus of any example herein, particularly Example 51, wherein the braided metal layer comprises braided metal wires.

### Example 53

The delivery apparatus of any example herein, particularly Example 52, wherein the metal wires have a diameter that is one or more of at least 0.002 inches, at least 0.003 inches, at least 0.004 inches, at least 0.005 inches, and at most 0.006 inches.

### Example 54

The delivery apparatus of any example herein, particularly any one of Examples 52-53, wherein the metal wires are braided with a pick density that is one or more of at least 10 picks per inch (ppi), at least 20 ppi, at least 30 ppi, at least 40 ppi, at least 50 ppi, at most 60 ppi, at most 45 ppi, at most 35 ppi, at most 25 ppi, at most 15 ppi, 10-25 ppi, 20-35 ppi, 30-45 ppi, 40-60 ppi, 10-35 ppi, 20-45 ppi, 30-60 ppi, 10-45 ppi, and 20-60 ppi.

### Example 55

The delivery apparatus of any example herein, particularly any one of Examples 51-54, wherein the metal wires are braided with a pick density that varies along a length of the braided metal layer.

### Example 56

The delivery apparatus of any example herein, particularly any one of Examples 51-54, wherein the metal wires are braided with a first pick density in the distal region of the first shaft and with a second pick density that is smaller than the first pick density in the proximal region of the first shaft.

### Example 57

The delivery apparatus of any example herein, particularly Example 56, wherein the first pick density is 30-45 ppi and the second pick density is 10-25 ppi.

### Example 58

The delivery apparatus of any example herein, particularly any one of Examples 51-57, wherein the braided metal layer terminates short of a proximal end of the first shaft by a braided layer terminal offset that is one or more of at least 0.1 inches, at least 0.5 inches, at least 1 inch, at least 2 inches, at least 3 inches, at least 4 inches, at most 5 inches, at most 3.5 inches, at most 2.5 inches, at most 1.5 inches, at most 0.75 inches, at most 0.3 inches, 0.1-0.75 inches, 0.5-1.5 inches, 1-2.5 inches, 2-3.5 inches, 3-5 inches, 0.1-1.5 inches, 0.5-2.5 inches, 1-3.5 inches, 2-5 inches, 0.1-2.5 inches, 0.5-3.5 inches, 1-5 inches, 0.1-2.5 inches, 0.5-3.5 inches, 1-5 inches, 0.1-3.5 inches 0.5-5 inches, and 0.1-5 inches.

### Example 59

The delivery apparatus of any example herein, particularly any one of Examples 51-57, wherein the braided metal layer terminates at a proximal end of the first shaft.

### Example 60

The delivery apparatus of any example herein, particularly any one of Examples 46-59, wherein the first shaft comprises a first shaft body surrounding the first shaft liner and defining an external surface of the first shaft.

### Example 61

The delivery apparatus of any example herein, particularly Example 60, wherein the first shaft body forms a portion of each of the first flex portion and the second flex portion, wherein the first shaft body has the first flex portion hardness within the first flex portion, and wherein the first shaft body has the second flex portion hardness within the second flex portion.

### Example 62

The delivery apparatus of any example herein, particularly any one of Examples 60-61, wherein the first shaft body forms a portion of the distal tip portion, and wherein the first shaft body has the distal tip portion hardness within the distal tip portion.

### Example 63

The delivery apparatus of any example herein, particularly any one of Examples 45-62, wherein the first shaft lumen has a lumen inner diameter that is one or more of at least 0.10 inches, at least 0.15 inches, at least 0.20 inches, at least 0.25 inches, at least 0.30 inches, at most 0.35 inches, at most 0.27 inches, at most 0.22 inches, at most 0.17 inches, at most 0.12 inches, 0.10-0.17 inches, 0.15-0.22 inches, 0.20-0.27 inches, 0.25-0.35 inches, 0.10-0.22 inches, 0.15-0.27 inches, 0.20-0.35 inches, 0.10-0.27 inches, 0.15-0.35 inches, and 0.10-0.35 inches.

### Example 64

The delivery apparatus of any example herein, particularly any one of Examples 45-63, wherein the first shaft lumen has a lumen proximal inner diameter in at least a portion of the distal region and a lumen distal inner diameter in at least a portion of the distal tip portion, and wherein the lumen distal inner diameter is greater than the lumen proximal inner diameter.

### Example 65

The delivery apparatus of any example herein, particularly Example 64, wherein the lumen proximal inner diameter is one or more of at least 0.10 inches, at least 0.15 inches, at least 0.20 inches, at least 0.25 inches, at least 0.30 inches, at most 0.35 inches, at most 0.27 inches, at most 0.22 inches, at most 0.17 inches, at most 0.12 inches, 0.10-0.17 inches, 0.15-0.22 inches, 0.20-0.27 inches, 0.25-0.35 inches, 0.10-0.22 inches, 0.15-0.27 inches, 0.20-0.35 inches, 0.10-0.27 inches, 0.15-0.35 inches, and 0.10-0.35 inches.

### Example 66

The delivery apparatus of any example herein, particularly any one of Examples 64-65, wherein the lumen distal inner diameter is one or more of at least 0.10 inches, at least 0.15 inches, at least 0.20 inches, at least 0.25 inches, at least 0.30 inches, at most 0.35 inches, at most 0.27 inches, at most 0.22 inches, at most 0.17 inches, at most 0.12 inches, 0.10-0.17 inches, 0.15-0.22 inches, 0.20-0.27 inches, 0.25-0.35 inches, 0.10-0.22 inches, 0.15-0.27 inches, 0.20-0.35 inches, 0.10-0.27 inches, 0.15-0.35 inches, and 0.10-0.35 inches.

### Example 67

The delivery apparatus of any example herein, particularly any one of Examples 64-66, wherein the first shaft comprises a lumen shoulder, wherein the first shaft lumen has the lumen proximal inner diameter on a proximal side of the lumen shoulder, and wherein the first shaft lumen as the lumen distal inner diameter on a distal side of the lumen shoulder.

### Example 68

The delivery apparatus of any example herein, particularly any one of Examples 1-67, wherein the delivery apparatus is sterilized.

### Example 69

A shaft comprising: a distal region; a proximal region; and a lumen configured to receive a second shaft therein, wherein the distal region of the shaft comprises: a distal tip portion configured to engage an implant apparatus supported by the second shaft and having a distal tip portion hardness; a first flex portion extending on a proximal side of the distal tip portion and having a first flex portion hardness that is less than the distal tip portion hardness; a second flex portion extending on a proximal side of the first flex portion and having a second flex portion hardness that is greater than the first flex portion hardness; and a distal region bulk portion extending between the second flex portion and the proximal region and having a distal region bulk hardness that is greater than the second flex portion hardness.

### Example 70

The shaft of any example herein, particularly Example 69, wherein the shaft is the first shaft of any example herein, particularly any one of Examples 1-68.

### Example 71

The shaft of any example herein, particularly any one of Examples 69-70, wherein the shaft is sterilized.

### Example 72

A delivery apparatus comprising: the shaft of any example herein, particularly any one of Examples 69-71; and the second shaft disposed radially interiorly of the shaft.

### Example 73

The delivery apparatus of any example herein, particularly Example 72, wherein the delivery apparatus is sterilized.

### Example 74

A delivery assembly comprising: a guide catheter comprising a guide catheter handle and a guide catheter shaft extending distally from the guide catheter handle; and a delivery apparatus comprising a delivery apparatus handle and a delivery shaft extending distally from the delivery apparatus handle, wherein the delivery shaft is configured to be advanced through the guide catheter shaft to deliver a prosthetic implant to an implantation site within a patient's body, wherein the guide catheter handle comprises one or more articulation members configured to be adjusted by a user to articulate a distal end portion of the guide catheter shaft within the patient's body, and wherein the delivery apparatus handle lacks articulation members for articulating a distal end portion of the delivery shaft.

### Example 75

The delivery assembly of any example herein, particularly Example 74, wherein the delivery shaft is the delivery shaft of any example herein, particularly any one of Examples 1-68.

### Example 76

The delivery assembly of any example herein, particularly any one of Examples 74-75, further comprising a docking device delivery apparatus configured to deliver a docking device to the implantation site, wherein the docking device is configured to at least partially anchor the prosthetic implant at the implantation site.

### Example 77

The delivery assembly of any example herein, particularly Example 76, wherein the docking device delivery apparatus comprises a docking device delivery handle, a docking device delivery shaft extending distally from the handle and configured to be advanced through the guide catheter shaft, and a pusher assembly configured to deploy the docking device at the implantation site.

### Example 78

The delivery assembly of any example herein, particularly any one of Examples 74-77, further comprising the prosthetic implant.

### Example 79

The delivery assembly of any example herein, particularly Example 78, wherein the prosthetic implant comprises a prosthetic heart valve, and wherein the implantation site is a native valve of the patient's heart.

### Example 80

The delivery assembly of any example herein, particularly any one of Examples 74-79, wherein the delivery assembly is sterilized.

### Example 81

A delivery apparatus comprising: a handle; and a delivery shaft extending distally from the handle and comprising: a first shaft comprising a distal region and a proximal region; and a second shaft disposed radially interiorly of the first shaft, wherein the first shaft is configured to translate relative to the second shaft along a longitudinal axis of the delivery shaft, wherein the second shaft comprises a distal end region configured to support a prosthetic implant thereon, and wherein the handle is free of articulation members for bending a distal end portion of the delivery shaft.

### Example 82

The delivery apparatus of any example herein, particularly Example 81, wherein the distal region of the first shaft comprises: a distal tip portion configured to engage an implant apparatus supported by the second shaft and having a distal tip portion hardness; a first flex portion extending on a proximal side of the distal tip portion and having a first flex portion hardness that is less than the distal tip portion hardness; a second flex portion extending on a proximal side of the first flex portion and having a second flex portion hardness that is greater than the first flex portion hardness; and a distal region bulk portion extending between the second flex portion and the proximal region and having a distal region bulk hardness that is greater than the second flex portion hardness.

### Example 83

The delivery apparatus of any example herein, particularly any one of Examples 81-82, wherein the delivery shaft is the delivery shaft of any example herein, particularly any one of Examples 1-68.

### Example 84

The delivery apparatus of any example herein, particularly any one of Examples 81-83, wherein the delivery apparatus is sterilized.

### Example 85

A delivery assembly comprising: a guide catheter comprising a guide catheter handle and a guide catheter shaft extending distally from the guide catheter handle; and the delivery apparatus of any example herein, particularly any one of Examples 81-84, wherein the delivery shaft is configured to be advanced through the guide catheter shaft to deliver a prosthetic implant to an implantation site within a patient's body, and wherein the guide catheter handle comprises one or more articulation members configured to be adjusted by a user to articulate a distal end portion of the guide catheter shaft within the patient's body.

### Example 86

The delivery assembly of any example herein, particularly Example 85, further comprising a docking device delivery apparatus configured to deliver a docking device to the implantation site, wherein the docking device is configured to at least partially anchor the prosthetic implant at the implantation site.

### Example 87

The delivery assembly of any example herein, particularly Example 86, wherein the docking device delivery apparatus comprises a docking device delivery handle, a docking device delivery shaft extending distally from the handle and configured to be advanced through the guide catheter shaft, and a pusher assembly configured to deploy the docking device at the implantation site.

### Example 88

The delivery assembly of any example herein, particularly any one of Examples 85-87, further comprising the prosthetic implant.

### Example 89

The delivery assembly of any example herein, particularly any one of Examples 85-88, wherein the prosthetic implant comprises a prosthetic heart valve, and wherein the implantation site is a native valve of the patient's heart.

### Example 90

The delivery assembly of any example herein, particularly any one of Examples 85-90, wherein the delivery assembly is sterilized.

### Example 91

A method comprising sterilizing the device, apparatus, and/or assembly of any Example herein.

### Example 92

A method comprising using the device, apparatus, and/or assembly of any Example herein to deliver a prosthetic implant to an implantation site within a patient's body.

The features described herein with regard to any example can be combined with other features described in any one or more of the other examples, unless otherwise stated. For example, any one or more of the features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus comprising:
a delivery shaft comprising:
a first shaft comprising a distal region and a proximal region; and
a second shaft disposed radially interiorly of the first shaft,
wherein the first shaft is configured to translate relative to the second shaft along a longitudinal axis of the delivery shaft, wherein the second shaft comprises a distal end region configured to support a prosthetic implant thereon, and wherein the distal region of the first shaft comprises:
a distal tip portion configured to engage an implant apparatus supported by the second shaft and having a distal tip portion hardness;
a first flex portion extending on a proximal side of the distal tip portion and having a first flex portion hardness that is less than the distal tip portion hardness;
a second flex portion extending on a proximal side of the first flex portion and having a second flex portion hardness that is greater than the first flex portion hardness; and
a distal region bulk portion extending between the second flex portion and the proximal region and having a distal region bulk hardness that is greater than the second flex portion hardness.

2. The delivery apparatus of claim 1, wherein the distal tip portion has a distal tip portion length that 0.1-1 inches, wherein the first flex portion has a first flex portion length that is 1-3.5 inches, wherein the second flex portion has a second flex portion length that is 0.5-1.5 inches, and wherein the distal region bulk portion has a distal region bulk portion length that is 35-50 inches.

3. The delivery apparatus of claim 1, wherein the distal tip portion comprises a radiopaque marker.

4. The delivery apparatus of claim 1, wherein the distal tip portion hardness corresponds to a durometer value that is 50-60 D, wherein the first flex portion hardness corresponds to a durometer value that is 20-35 D, wherein the second flex portion hardness corresponds to a durometer value that is 50-65 D, and wherein the distal region bulk hardness corresponds to a durometer value that is 65-80 D.

5. The delivery apparatus of claim 1, wherein the proximal region has a proximal region outer diameter that is smaller than a distal region outer diameter of the distal region.

6. The delivery apparatus of claim 5, wherein the distal region outer diameter is 0.2-0.4 inches, and wherein the proximal region has a proximal region outer diameter that is 0.15-0.32 inches.

7. The delivery apparatus of claim 5, wherein the first shaft comprises a transition region in which an outer diameter of the first shaft varies between the proximal region outer diameter and the distal region outer diameter.

8. The delivery apparatus of claim 1, wherein the first shaft comprises a first shaft lumen that receives the second shaft therein and a first shaft liner that at least partially defines the first shaft lumen.

9. The delivery apparatus of claim 8, wherein the first shaft liner has a first shaft liner thickness that 0.005-0.012 inches.

10. The delivery apparatus of claim 8, wherein the first shaft comprises a braided metal layer surrounding the first shaft liner.

11. The delivery apparatus of claim 10, wherein the braided metal layer comprises braided metal wires that are braided with a pick density that is 10-45 ppi.

12. The delivery apparatus of claim 11, wherein the braided metal layer terminates short of a proximal end of the first shaft by a braided layer terminal offset that is 0.1-0.75 inches.

13. The delivery apparatus of claim 11, wherein the braided metal layer terminates at a proximal end of the first shaft.

14. A shaft comprising:
a distal region;
a proximal region; and
a lumen configured to receive a second shaft therein,
wherein the distal region of the shaft comprises:
a distal tip portion configured to engage an implant apparatus supported by the second shaft and having a distal tip portion hardness;
a first flex portion extending on a proximal side of the distal tip portion and having a first flex portion hardness that is less than the distal tip portion hardness;
a second flex portion extending on a proximal side of the first flex portion and having a second flex portion hardness that is greater than the first flex portion hardness; and
a distal region bulk portion extending between the second flex portion and the proximal region and having a distal region bulk hardness that is greater than the second flex portion hardness.

15. The shaft of claim 14, wherein the distal tip portion hardness corresponds to a durometer value that is 50-60 D, wherein the first flex portion hardness corresponds to a durometer value that is 20-35 D, wherein the second flex portion hardness corresponds to a durometer value that is 50-65 D, and wherein the distal region bulk hardness corresponds to a durometer value that is 65-80 D.

16. The shaft of claim 14, wherein the distal region further comprises a third flex portion extending on a proximal side of the second flex portion and having a third flex portion hardness that is greater than the second flex portion hardness.

17. The shaft of claim 16, wherein the distal region bulk portion extends between the third flex portion and the proximal region, and wherein the distal region bulk hardness is greater than the third flex portion hardness.

18. The shaft of claim 16, wherein the third flex portion hardness corresponds to a durometer value that is 40-80 D.

19. The shaft of claim 16, wherein the distal region further comprises a fourth flex portion extending on a proximal side of the third flex portion and having a fourth flex portion hardness that is greater than the third flex portion hardness.

20. A delivery assembly comprising:
a guide catheter comprising a guide catheter handle and a guide catheter shaft extending distally from the guide catheter handle; and
a delivery apparatus comprising a delivery apparatus handle and a delivery shaft extending distally from the delivery apparatus handle,
wherein the delivery shaft comprises:
a distal region;
a proximal region; and
a lumen configured to receive a second shaft therein,
wherein the distal region of the shaft comprises:
a distal tip portion configured to engage an implant apparatus supported by the second shaft and having a distal tip portion hardness;
a first flex portion extending on a proximal side of the distal tip portion and having a first flex portion hardness that is less than the distal tip portion hardness;
a second flex portion extending on a proximal side of the first flex portion and having a second flex portion hardness that is greater than the first flex portion hardness; and
a distal region bulk portion extending between the second flex portion and the proximal region and having a distal region bulk hardness that is greater than the second flex portion hardness,
wherein the delivery shaft is configured to be advanced through the guide catheter shaft to deliver a prosthetic implant to an implantation site within a patient's body, wherein the guide catheter handle comprises one or more articulation members configured to be adjusted by a user to articulate a distal end portion of the guide catheter shaft within the patient's body, and wherein the delivery apparatus handle lacks articulation members for articulating a distal end portion of the delivery shaft.
